(19) United States
(12) Patent Application Publication
(10) Pub. No.: US 2026/0148825 A1
(43) Pub. Date: May 28, 2026

(54) PREDICTING TREATMENT EFFICACY BY ANALYZING RELATIVE ALLELIC EXPRESSION

(71) Applicant: Foundation Medicine, Inc., Cambridge, MA (US)

(72) Inventors: Meagan Montesion, Cambridge, MA (US); Dexter Jin, Saugus, MA (US)

(21) Appl. No.: 19/402,700

(22) Filed: Nov. 26, 2025

Related U.S. Application Data
(60) Provisional application No. 63/725,995, filed on Nov. 27, 2024.

(51) Int. Cl. G16H 20/17; C12N 15/10; C12Q 1/6855; C12Q 1/6874; G16B 20/10; G16B 25/10; G16H 15/00; G16H 50/20

(52) U.S. Cl. CPC G16H 20/17; C12N 15/1096; C12Q 1/6855; C12Q 1/6874; G16B 20/10; G16B 25/10; G16H 15/00; G16H 50/20; C12Q 2600/158

(57) ABSTRACT

Techniques for identifying various health-related conditions based on allelic expression are described. In an example method, sequence read data of a sample obtained from the subject is identified. The sequence read data is indicative of RNA molecules in the sample. The example method further comprises determining the expression of alleles of a gene, determining a discrepancy between the expression of the alleles of the gene, and predicting a health-related condition, such as a treatment efficacy, based on the discrepancy between the expression of the alleles of the gene.

### CROSS-REFERENCE TO RELATED APPLICATION

This application claims priority to U.S. Provisional App. No. 63/725,995, which was filed on Nov. 27, 2024 and is incorporated by reference herein in its entirety.

### BACKGROUND

Clinicians rely on various types of diagnostic tests in order to categorize cancers. For example, clinicians are able to assess cancer types by performing physical exams, medical imaging, histological studies, and other diagnostic analyses. In general, different types of cancer treatments are effective at treating different types of cancers. Accordingly, a clinician may prescribe or administer a cancer treatment to a patient based on the type of cancer cells identified in the patient's body.

Immunotherapies can treat the cancer cells by activating and/or suppressing the immune system of the patient. For instance, particular immune checkpoint inhibitors can be used to induce T cells to destroy PD-L1-expressing cancer cells in the patient's body. A clinician may predict whether a PD-L1-targeting immunotherapy will be effective in treating the cancer cells by determining whether the cancer cells express PD-L1 via immunohistochemistry (IHC). However, PD-L1 expression by the cancer cells may not be determinative of whether a PD-L1-targeting immunotherapy will be effective. For instance, in many cases, PD-L1-targeting immunotherapies are ineffective at treating PD-L1-positive cancer cells.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates an example environment for determining a health-related condition of a subject 102 based on allelic expression of one or more genes of the subject.

FIG. 2 illustrates an example environment for sequencing various RNA molecules.

FIG. 3 illustrates an example report summarizing predicted categories of a cancer of a subject.

FIG. 4 illustrates an example process for identifying a predicted effective treatment based on an allelic expression of a subject.

FIG. 5 illustrates one or more devices configured to perform various operations described herein.

FIG. 6 illustrates HLA genotyping concordance between DNA and RNA samples.

FIG. 7 illustrates that lower median HLA minor allele frequency (MAF) is seen in samples that are HLA LOH positive.

FIG. 8 illustrates prevalence of HLA loss of function (LOF).

### DETAILED DESCRIPTION

Various implementations of the present disclosure relate to techniques for predicting various health-related conditions, such as treatment efficacy, by analyzing relative expression of alleles of a gene. In various cases, nucleic acid molecules are obtained from a subject. In some cases, the nucleic acid molecules include RNA obtained from a sample obtained from the subject, such as a tissue biopsy sample or a liquid biopsy sample. Sequence read data is generated by sequencing the nucleic acid molecules. In some cases, proteins and/or peptides are obtained from the sample obtained from the subject. The sequence read data may be generated by sequencing the proteins and/or peptides. The expression of the alleles of the gene can be determined, for instance, based on the number of reads in the sequence read data corresponding to each of the alleles of the gene. The relative expression of a particular allele of the gene is, in various cases, determined based on a ratio of the expression of the particular allele of the gene to the expression of all of the alleles of the gene. In various examples, the relative expression of alleles of more than one gene may be analyzed to predict the health-related condition.

Various types of health-related conditions can be predicted using various techniques described herein. In various cases, these techniques are used to determine whether the subject has a cancer type and/or a cancer subtype. In some cases, these techniques are used to determine whether a therapy is predicted to treat a pathological condition (e.g., cancer) of the subject. In various instances, these techniques are used to determine a prognosis (e.g., a predicted survivability) of the subject. In some examples, these techniques can be used to predict a tumor progression or whether the tumor is susceptible to a particular treatment.

Implementations of the present disclosure provide significant improvements to the technical field of cancer diagnosis, management, and treatment. Using current technologies, allelic loss of heterozygosity is typically categorized by performing a tissue biopsy on a potential tumor and determining genomic expression of one or more alleles of a gene. Utilizing RNA and/or proteomic expression of alleles of the gene can greatly enhance the predictive accuracy of whether immunotherapies, and other types of cancer treatments, will be effective when administered to the subject. For instance, genomic analysis (e.g., DNA analysis) can indicate whether the subject has a homozygous genotype (e.g., two alleles of a gene that are substantially identical) or a heterozygous genotype (e.g., two alleles of a gene that are different) associated with a gene. However, the RNA and/or proteomic expression can inform how that gene is expressed. For instance, the RNA expression of a gene can indicate loss of function of an allele or of the gene. Accordingly, a clinician relying on various predictions described herein is less likely to prescribe or administer a treatment that will be ineffective. As a result, the subject is less likely to suffer the side effects and financial burdens of treatments that have minimal therapeutic benefit. Further, the clinician is less likely to present an inaccurate prognosis to the patient. Inaccurate prognoses and ineffective treatments can cause significant emotional hardship, side effects, and financial burden on the patient, and can be avoided using various techniques described herein.

Various analyses described herein cannot be performed in the human mind, or by pen and paper. For example, the sequence read data may represent numerous (e.g., thousands) of bases to be analyzed. In various cases, it would be impossible to manually or mentally identify expression of alleles, including variants of the alleles, based on the sequence read data. Moreover, it would not be possible for a human to generate an accurate assessment of various types of health-related conditions, including a predicted effectiveness of a treatment or a predicted tumor progression, based on the expression of multiple alleles of multiple genes. Particular implementations of the present disclosure are fundamentally tied to computer technology, and do not represent mere automation of processes that are performed manually.

### Example Definitions

As used herein, the terms “deoxyribonucleic acid,” “DNA,” “DNA molecule,” and their equivalents, may refer to a polymer of nucleotides (also referred to as “nucleobases”) containing deoxyribose. The nucleotides in DNA include cytosine (C), guanine (G), adenine (A), and thymine (T). Each DNA nucleotide includes a deoxyribose and a phosphate group. An example single-stranded DNA (ssDNA) molecule includes a chain of covalently bonded DNA nucleotides. In the example ssDNA molecule, the phosphate group of the mth nucleotide is covalently bonded to the deoxyribose of the (m−1)th nucleotide, wherein m is a positive integer greater than 2 and less than or equal to the number of DNA nucleotides in the chain. In various examples, DNA is double-stranded and includes two ssDNA molecules that are complementary to one another and coiled around each other in a double helix form. The nucleotides of one ssDNA molecule are hydrogen bonded to the nucleotides of the other ssDNA molecule. In particular, the pyrimidines (A and T) hydrogen bond to each other, and the purines (C and G) hydrogen bond to each other.

As used herein, the terms “ribonucleic acid,” “RNA,” “RNA molecule,” and their equivalents, may refer to a polymer of nucleotides containing ribose. The nucleotides in RNA include cytosine (C), guanine (G), adenine (A), and uracil (U). Each RNA nucleotide includes a ribose and a phosphate group. In an example RNA molecule, the phosphate group of the nth nucleotide is covalently bonded to the ribose of the (n−1)th nucleotide, wherein n is a positive integer greater than 2 and less than or equal to the number of RNA nucleotides in the chain. Messenger RNA (mRNA) is a type of RNA molecule that is synthesized (or “transcribed”) by RNA polymerase (an enzyme) to be complementary to a gene encoded in a DNA sequence, and is also used by a ribosome to synthesize a polypeptide or protein. An mRNA is therefore an example of a “coding RNA.” In various cases, intron sequences are removed from an mRNA via a process known as “RNA splicing.” MicroRNA (“miRNA”) are single-stranded RNA molecules that perform post-transcriptional gene expression regulation. For instance, a miRNA may bind to a complementary mRNA molecule, thereby cleaving, destabilizing, or otherwise preventing the mRNA molecule from being translated into a polypeptide or protein by a ribosome. In various examples, a miRNA has a length in a range of 21 to 23 RNA nucleotides. As used herein, the terms “non-coding RNA” may refer to a type of RNA that is not translated into a protein. Examples of non-coding RNA include miRNA, transfer RNA (tRNA), and ribosomal RNA (rRNA). The term “functional RNA,” and its equivalents, may refer to any RNA molecule that impacts a biological process. For instance, functional RNA may include mRNA, miRNA, tRNA, rRNA, and the like.

As used herein, the term “base,” and its equivalents, may refer to a monomer of a polymer. For example, a base of DNA or RNA is a nucleotide.

As used herein, the term “base pair,” and its equivalents, may refer to a pair of complementary DNA nucleotides, which are hydrogen-bonded to one another in a double-stranded DNA molecule. For example, a base pair includes a first base in a first ssDNA and a second base in a second ssDNA, wherein the first and second bases are complementary and hydrogen-bonded to one another.

As used herein, the terms “nucleotide,” “nucleobase,” “nucleic acid,” “nucleic acid molecule,” and their equivalents, may refer to an organic molecule that includes a nitrogenous base, a sugar, and a phosphate group. In various cases, a nucleotide is a monomer of DNA or RNA. A nucleotide, for instance, is a chemical structure.

As used herein, the terms “3′ end,” “3-prime end,” and their equivalents, may refer to a terminus of a single-stranded nucleotide polymer that includes a base whose third carbon in its deoxyribose or ribose is bound to a hydroxyl group while being unbound to another base.

As used herein, the terms “5′ end,” “5-prime end,” and their equivalents, may refer to a terminus of a single-stranded nucleotide polymer that includes a base whose fifth carbon in its deoxyribose or ribose ring is unbound to another base. In some cases, the fifth carbon is bound to a phosphate group.

As used herein, the “length” of a polymer refers to a number of covalently bonded monomers that are included in the polymer. For instance, the length of a DNA molecule may be the number of covalently bonded nucleotides in at least one strand of the DNA molecule and/or the number of base pairs in the DNA molecule. In various examples, the length of an RNA molecule may be the number of covalently bonded nucleotides in the RNA molecule.

As used herein, the term “gene,” and its equivalents, refers to a sequence of DNA nucleotides that is transcribed into a functional RNA. The functional RNA, for instance, is RNA that is translated into a polypeptide or protein (e.g., mRNA) or that has some other biological function (e.g., miRNA, tRNA, etc.). A gene is “expressed” when it is used as a template to generate a functional RNA. A subject, for instance, has numerous genes contained in the subject's genome. A gene may include both introns and exons. As used herein, the term “intron,” and its equivalents, may refer to a subset of DNA nucleotides in a gene that is not used to code for any functional RNA that is expressed by the organism. As used herein, the term “exon,” and its equivalents, may refer to a subset of DNA nucleotides in a gene that is used to code for a functional RNA. For instance, an exon may encode a polypeptide or protein that is expressed by the organism. In various examples, a gene can be represented in data (e.g., as data representative of the sequence of DNA nucleotides in the gene) or as a chemical structure (e.g., as the sequence of DNA nucleotides itself).

As used herein, the term “allelic expression,” and its equivalents refers to the expression of one or more alleles of a gene. For instance, allelic expression may refer to at least one of a DNA expression, an RNA expression, or a protein expression of each of the one or more alleles of a gene. As used herein, the terms “heterozygous expression,” “heterozygous,” and their equivalents, refer to expression of two alleles of a gene, wherein the two alleles are different. As used herein, the terms “homozygous expression,” “homozygous,” and their equivalents refer to expression of two alleles of a gene, wherein the two alleles are substantially identical. As used herein, the term “loss of heterozygosity,” and its equivalents refers to loss of expression of at least one allele of a gene. Loss of heterozygosity, in some cases, may be associated with homozygous expression.

As used herein, the term “true expression,” “protein expression,” and its equivalents, refer to the protein expression of one or more genes.

As used herein, the term “genome,” and its equivalents, refers to the aggregate of genes of a subject. In various cases, a genome represents the sequences of several linear DNA molecules that are present in a subject's chromosomes. A “reference genome” refers to an aggregation of genes of one or more reference subjects. In various cases, a genome is represented in data.

As used herein, the terms “pangenome,” “pan-genome,” “supragenome,” and their equivalents, refers to an aggregate set of genes from multiple subgroups (e.g., strains) within a population (e.g., a clade) of subjects. A pangenome, for example, indicates genes that are present in all subjects within the population, as well as genes that are present in some of the subjects of the population. A pangenome is represented in data, for instance.

As used herein, the term “transcriptome,” and its equivalents, refers to the aggregate of RNA sequences of a subject. In some cases, a transcriptome is limited to mRNA sequences. In various examples, a transcriptome is represented in data.

As used herein, the term “genomic DNA,” “gDNA,” “chromosomal DNA,” and their equivalents, may refer to DNA molecules that are obtained from a chromosome and/or nucleus of a cell.

As used herein, the terms “nucleic acid fragment,” “fragment,” and their equivalents, may refer to nucleic acid molecules (e.g., RNA molecules and/or DNA molecules) that are excised and/or broken off from a larger nucleic acid molecule.

As used herein, the terms “cell-free DNA,” “cfDNA,” “cell-free RNA,” “cfRNA,” and their equivalents, may refer to nucleic acid fragments (e.g., DNA fragments and/or RNA fragments) that are non-encapsulated and obtained outside of cells within a sample (e.g., a liquid biopsy sample).

As used herein, the terms “circulating tumor DNA,” “ctDNA,” “circulating tumor RNA,” “ctRNA” and their equivalents, may refer to a cfDNA molecule or a cfRNA molecule that originates from a cancer cell.

As used herein, the terms “end motif,” “terminal sequences,” and their equivalents, may refer to a sequence of nucleotides extending from a 3′ or 5′ end of a DNA or RNA molecule. In various cases, the end motif is shorter than a length of the DNA or RNA molecule. For example, the end motif may have a length in a range of 5 to 30 bases or base pairs, a range of 3 to 30 bases or base pairs, or a range of 1 to 30 base pairs.

As used herein, the term “promoter,” and its equivalents, may refer to a portion of a DNA molecule that binds one or more proteins in order to initiate transcription of a gene. For example, the promotor is located “upstream” of the gene. For example, the promotor is located between the 5′ end of the DNA molecule and the gene. A promotor may include one or more binding sites for RNA polymerase, and/or one or more transcription factor binding sites. In some examples, a promotor includes one or more CpG islands. A promoter, for instance, includes a transcription start site.

As used herein, the terms “CpG island,” “CGI,” “CpG site,” and their equivalents, may refer to a continuous portion of a DNA molecule whose sequence includes greater than a threshold amount (e.g., greater than 50%) of G-C base pairs.

As used herein, the term “enhancer,” and its equivalents, may refer to a portion of a DNA molecule that binds one or more proteins in order to increase the chance that a gene will be transcribed. For instance, an enhancer includes one or more transcription factor binding sites. In various cases, an enhancer includes one or more CpG islands.

As used herein, the term “cancer,” and its equivalents, may refer to a condition of a subject in which particular cells (referred to as “cancer cells”) divide uncontrollably in the subject's body. In some cases, a cancer is characterized by a location or tissue type from which the cancer cells originated. In some examples, a cancer is characterized by a location or tissue type in which the cancer cells are located.

As used herein, the terms “tumor,” “neoplasm,” and their equivalents, may refer to a mass of tissue including cancer cells.

As used herein, the terms “tissue of origin,” “tissue origin,” and their equivalents, refers to a differentiated type of tissue from which cancer cells in the body of a subject began dividing uncontrollably in the subject's body.

As used herein, the terms “liquid biopsy,” “fluid biopsy,” and their equivalents, may refer to a process of obtaining a fluid sample from a subject's body. The sample, for instance, can be referred to as a “liquid biopsy sample.” Examples of fluids that are sampled from the body include blood, plasma, cerebrospinal fluid, sputum, stool, urine, lymphatic fluid, and saliva.

As used herein, the term “tissue biopsy,” and its equivalents, may refer to a process of obtaining a sample of cells from a subject's body. A tissue biopsy, in various cases, is performed by cutting a mass of cells from the subject's body. For instance, a tissue biopsy is a procedure performed by a surgeon, interventional radiologist, interventional cardiologist, or other specialized clinician. The term “tissue” or “tissue biopsy sample” can be used to refer to the sample of cells obtained using a tissue biopsy.

As used herein, the term “subject,” and its equivalents, may refer to a human or non-human animal. A subject that is receiving care from at least one care provider may be referred to as a “patient.”

As used herein, the terms “machine learning,” “ML,” “computer learning,” “artificial intelligence,” and their equivalents, may refer to the use of a computing devices to learn patterns in training data. The process of learning these patterns may be referred to as “training.” In particular cases, one or more computing devices may perform machine learning by executing a machine learning model. As used herein, the terms “machine learning model,” “ML model,” and their equivalents, may refer to data encoding instructions that, when executed by at least one computing device, causes the at least one computing device to learn patterns in training data by optimizing one or more metrics, values, or other types of parameters. After training, an ML model, when executed by at least one computing device, causes the at least one computing device to utilize the optimized parameters in order to perform one or more tasks.

As used herein, the term “variant,” and its equivalents, may refer to a difference between a subject's genetic sequence and a reference sequence. For instance, a variant may correspond to a difference between one or more nucleotides in a genome of a subject and one or more corresponding nucleotides in at least one reference genome or pangenome. A variant may be characterized by its identity (e.g., what nucleotides are different), its position (e.g., where are the nucleotides located in the genome, what chromosome contains the nucleotides, what gene contains the nucleotides, etc.), its length (e.g., how many nucleotides are different from the reference sequence), its type (e.g., substitution, insertion, deletion, copy number alternation, rearrangement of fusion, etc.), and other features that indicates its significance and/or relevance. In some cases, a variant represents any apparent alteration in a sequence that has been read from a nucleic acid molecule with respect to the reference sequence, such as reads cleaved by restriction enzymes (RE). In various examples, a variant can be represented in data (e.g., by data characterizing the variant) or as a chemical structure (e.g., the nucleotides themselves). As used herein, the term “mutation,” and its equivalents, may refer to a change in a gene.

As used herein, the term “substitution,” and its equivalents, can refer to a nucleotide in a subject sequence that is different than an equivalent nucleotide (e.g., a nucleotide at the same position) in a reference sequence.

As used herein, the term “insertion,” and its equivalents, can refer to a nucleotide in a subject sequence that is added with respect to a reference sequence.

As used herein, the term “deletion,” and its equivalents, can refer to the removal of a nucleotide from a nucleotide sequence.

As used herein, the terms “copy number alternation,” “CNA,” “copy number variation,” “CNV,” and their equivalents, can refer to a portion of a reference sequence that is repeated.

As used herein, the term “sequencing,” and its equivalents, may refer to a process of identifying the order and identity of monomers in a polymer chain, such as the order and identity of nucleotides in a DNA or RNA molecule. The terms “whole genome sequencing,” “WGS,” and their equivalents, may refer to the process of sequencing an entire genome of a subject, including the introns and exons of the genes of the subject. The term “whole exome sequencing,” and its equivalents, may refer to the process of sequencing all exomes of a subject. The term “targeted sequencing,” and its equivalents, may refer to the process of sequencing a portion of the genome of a subject, such as sequencing a single gene of the subject. Various techniques can be utilized to sequence a DNA or RNA molecule, such as massively parallel sequencing (MPS), nanopore sequencing, direct sequencing, Sanger sequencing, or next-generation sequencing. In various cases, sequencing is performed on physical molecules (e.g., RNA or DNA) and is used to generate data.

As used herein, the terms “massive parallel sequencing,” “massively parallel sequencing,” “MPS,” and their equivalents, may refer to a technique for simultaneously performing multiple reactions that can be used to identify the order and identity of monomers in multiple polymer chains. In particular cases, massive parallel sequencing can be performed using sequencing-by-synthesis on clonally amplified DNA molecules (e.g., complementary DNA (cDNA) molecules) that are located in spatially separated regions, which are individually monitored by sensors.

As used herein, the term “nanopore sequencing,” and its equivalents, may refer to a technique for identifying the order and identity of monomers in a polymer chain by transporting the polymer chain from a first space to a second space, wherein the first space and the second space are separated by a substrate, by directing the polymer chain through a small hole (known as a “nanopore”) embedded in the substrate, and monitoring a relative electrical signal (e.g., a voltage or current) between the first space and the second space.

As used herein, the term “sensor,” and its equivalents, may refer to a physical device or other apparatus that is configured to detect one or more detection signals.

As used herein, the term “detection signal,” and its equivalents, may refer to a physical signal that can be identified, characterized, or otherwise perceived by a sensor.

As used herein, the term “sequence read data,” and its equivalents, may refer to data that is indicative of an order and identity of monomers in a polymer, such as the order and identity of nucleotides in a DNA or RNA sequence. In various implementations, sequence read data is generated via a sequencing operation.

As used herein, the term “image,” and its equivalents, may refer to 2D or 3D array of data indicative of an array of pixels or voxels.

As used herein, the term “ligating,” and its equivalents, may refer to a process of joining two molecules together, for example, with a chemical bond.

As used herein, the terms “adapter,” “adaptor,” and their equivalents, may refer to an oligonucleotide that can be ligated to a target nucleic acid molecule. In various cases, an adapter prepares the target nucleic acid molecule for sequencing.

As used herein, the term “bait molecule,” and its equivalents, may refer to a nucleic acid molecule having a region that is complementary to a region of a target molecule (e.g., cfDNA). A bait molecule includes, for instance, a nucleic acid molecule that can hybridize to (i.e., is complementary to) a target molecule can be used to capture the target molecule. In some instances, the bait molecule is a capture oligonucleotide (or capture probe). In some instances, the bait molecule is suitable for solution phase hybridization to the target molecule. In some instances, the bait molecule is suitable for solid phase hybridization to the target molecule. In some instances, the bait molecule is suitable for both solution-phase and solid-phase hybridization to the target molecule. The design and construction of bait molecules is described in more detail in, e.g., International Patent Application Publication No. WO 2020/236941.

As used herein, the term “amplifying,” and its equivalents, may refer to a process of generating copies of a target molecule, such as a nucleic acid molecule.

As used herein, the term “hybridization,” and its equivalents, may refer to a process by which to complementary single-stranded nucleic acid molecules bind to one another, thereby forming a double-stranded nucleic acid molecule. In certain examples, the double-stranded nature of the nucleic acid molecule is maintained under stringent hybridization conditions. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C.

As used herein, the term “complementary,” and its equivalents, may refer to a state of two single-stranded nucleic acid molecules with respective sequences that cause the nucleic acid molecules to spontaneously hybridize to one another. One nucleic acid molecule, for instance, may have a sequence that causes each nucleic acid to hydrogen bond to a respective nucleic acid in the other nucleic acid molecule.

As used herein, the terms “therapy,” “treatment,” and their equivalents, may refer to a composition or process that can be used to remediate a health problem. Cancer therapies, for instance, include surgery, radiotherapy, chemotherapy, immunotherapy, cell-based therapies, and the like. Examples of immunotherapies include immune checkpoint inhibitors, a T-cell receptor (TCR) therapy, a TCR-bispecific molecule (e.g., bispecific T-cell engagers), a personalized cancer vaccine, chimeric antigen receptor T-cell (CAR T-cell) therapy, monoclonal antibodies, cytokine therapy, immunomodulators, antibody-drug conjugates, natural killer (NK) cell therapy, antibody-dependent cellular cytotoxicity (ADCC) enhancers, and the like. Examples of cancer therapies include abemaciclib (Verzenio), abiraterone acetate (Zytiga), acalabrutinib (Calquence), ado-trastuzumab emtansine (Kadcyla), afamitresgene autoleucel (Tecelra), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), Lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximabcmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecanhziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), and combinations thereof. Examples of cancer therapies also include targeted antibody-based therapies (antibody-drug conjugates, antibody-radioisotope conjugates, and targeted immune cell therapies (e.g., immune effector cells genetically modified to express a chimeric antigen receptor (CAR)).

As used herein, the term “treatment-responsive,” and its equivalents, may refer to a type of cancer cells that can be substantially killed using a predetermined type of therapy. For example, cancer cells of a subject may be responsive to a particular treatment if, after the subject is administered the treatment, the cancer cells are diminished by a particular progression level (e.g., radiographic progression level, marker-based progression level, such as prostate-specific antigen (PSA) progression, etc.). Accordingly, the responsiveness of the cells to the type of therapy may indicate the effectiveness of that therapy.

As used herein, the term “treatment-resistant,” and its equivalents, may refer to a type of cancer that cannot be substantially killed using a predetermined type of therapy.

As used herein, the term “metastasis profile,” and its equivalents, may refer to a propensity of a type of cancer to metastasize into one or more differentiated tumor types besides the cancer's tissue origin. In some implementations, the metastasis profile can further indicate the type of tissue in which the cancer can or is likely to metastasize.

As used herein, the term “clinical trial,” and its equivalents, may refer to a research study used to evaluate a hypothesis based on participation by one or more subjects. In various examples, a clinical trial can be used to assess the efficacy and/or safety of a proposed therapy. A clinical trial may be performed in furtherance of approval of a treatment by a regulatory authority (e.g., the United States Food & Drug Administration (FDA)).

### Description of Example Implementations

Various implementations of the present disclosure will now be described with reference to the accompanying Figures.

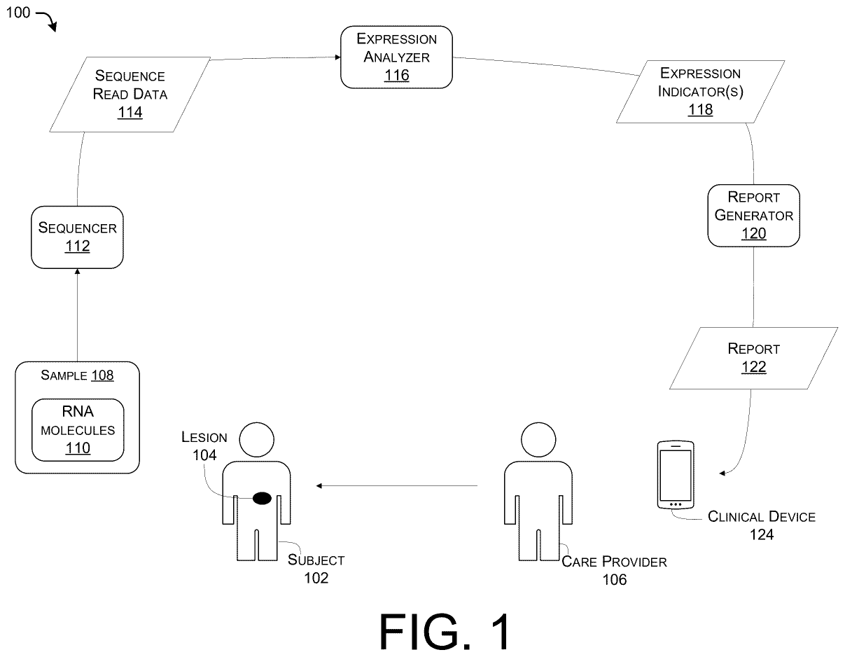

FIG. 1 illustrates an example environment 100 for determining a health-related condition of a subject 102 based on the relative allelic expression of one or more genes of the subject 102. In some cases, the subject 102 lacks any apparent disease or other pathological condition. For example, the subject 102 may present to a clinical environment for a medical assessment of the subject 102, such as an evaluation of the general health or well-being of the subject 102. In various cases, the subject 102 presents to the environment 100 as part of a screening assessment for a pathological condition (e.g., cancer). For instance, the subject 102 may schedule an appointment in the environment 100 based on an age or demographic of the subject 102, rather than in response to any symptom or suspected condition.

According to various examples, the subject 102 presents to a clinical environment with a lesion 104. In some examples, the subject 102 has one or more types of cancer, such as bladder cancer, breast cancer, colorectal cancer, a melanoma, non-small cell lung cancer (NSCLC), pancreatic cancer, or prostate cancer.

In some embodiments, the subject 102 has B cell cancer (multiple myeloma), a melanoma, breast cancer, lung cancer, NSCLC, small cell lung cancer (SCLC), bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, a carcinoid tumor, or combinations or metastases thereof.

In some embodiments, the subject 102 has acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), a non-small cell lung cancer KRAS (+/−G12C), a non-small cell lung cancer TMB-H, a non-small cell lung cancer MET exon 14 skipping, a non-small cell lung cancer ERBB2 inframe indel, a non-small cell lung cancer EGFR exon 20 indel, a neurotrophic tyrosine receptor kinase (NTRK)-positive cancer, ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In various implementations, the subject 102 has a non-cancer condition, such as a genetic disorder, diabetes, cardiac disease, hypertension, a metabolic disorder, a respiratory disease, an infectious disease, an autoimmune disease, a neurological disorder, arthritis, a pregnancy-related condition, organ dysfunction, organ failure, or another non-cancer condition.

In various cases, one or more care providers 106 (also referred to as “healthcare provider(s)”) is responsible for monitoring and/or treating the subject 102. The care provider(s) 106 may include one or more of a clinician, a surgeon, an anesthesiologist, a pathologist, a laboratory technician, or the like.

In various cases, one or more treatments exist for one or more subtypes of the condition. For instance, if cells in the lesion 104 express one or more proteins-of-interest, the treatment(s) may be able to specifically target the cells within the lesion 104 of the subject 102. For instance, the treatment(s) may include one or more immunotherapies, such as an immune checkpoint inhibitor, a T-cell receptor (TCR) therapy, a TCR-bispecific molecule (e.g., bispecific T-cell engagers), a personalized cancer vaccine, chimeric antigen receptor T-cell (CAR T-cell) therapy, monoclonal antibodies, a cytokine therapy, an immunomodulator, an antibody-drug conjugate, a natural killer (NK) cell therapy, an antibody-dependent cellular cytotoxicity (ADCC) enhancer, or the like. Proteins-of-interest include proteins targeted by the immunotherapy as well as proteins involved in the therapeutic response (e.g., an immune response, a pharmacological response, a hematological response, a physiological response, etc. of the subject 102 in response to the treatment(s)). For instance, an immune checkpoint inhibitor may be targeted to a particular checkpoint protein, such as PD-1, PDL-1, LAG-3, CTLA-4, TIM-3, A2AR, B7-H3, B7-H4, VISTA, BTLA, TIGIT, CD47, or the like. In various cases, the immune checkpoint inhibitor may block ligand binding to an inhibitory checkpoint receptor on T cells, thereby preventing the inhibition of T cell activation by the ligand. Increased T cell activation can facilitate a more effective immune response against cancer cells. However, T cells also interact with other proteins that are involved in the therapeutic response. For instance, human leukocyte antigen (HLA) molecules are responsible for presenting proteins and protein fragments from infected or cancerous cells to T cells to facilitate a targeted immune response. If the expression of one or more HLA molecules is altered, the immune checkpoint inhibitor may not be as effective in targeting the pathogenic cells (e.g., cancer cells) within the subject 102. Accordingly, if the cells in the lesion 104 fail to substantially express one or more of the proteins-of-interest, the treatment(s) may be ineffective. To avoid potential side effects of the treatment(s), as well as their associated costs, without effectively treating a pathological condition of the subject, the care provider 106 may initially categorize whether the cells in the lesion 104 and/or the subject 102 express the proteins-of-interest. Other examples of proteins-of-interest include HER2, MAGE-A3, NY-ESO-1, MART-1, WT1, PSMA, HPV E6, HPV E7, PSMA, PAP, CD proteins (CD19, CD20), BCMA, FAP, VEGF, EGFR, BCR-ABL, BRAF, and the like.

According to some implementations, the lesion 104 may be initially identified using a noninvasive technique. For example, the lesion 104 may be visualized using an imaging modality, such as ultrasound, x-ray, computed tomography (CT), magnetic resonance imaging (MRI), positron emission tomography (PET), single photon emission CT (SPECT), or any combination thereof. However, noninvasive imaging technologies may be unable to categorize whether the lesion 104 includes cells that express the proteins-of-interest.

In various implementations, the care provider 106 is unable to accurately identify the health-related condition of the subject 102 based solely on noninvasive diagnostic techniques. For instance, the care provider 106 cannot conclusively determine whether the cells of the subject 102 express the proteins-of-interest and whether the subject 102 will respond to a particular treatment based on noninvasive diagnostic techniques. In various examples, the care provider 106 may be unable to identify a characteristic of a subject presenting with a disease (e.g., cancer), wherein the characteristic is determinative of, or at least correlated with, an effectiveness of at least one therapy at treating the disease, an ineffectiveness of at least one therapy at treating the disease, a survivability (e.g., a likelihood that the subject will survive by a predetermined date or time), an expected quality of life, at least one predetermined symptom, at least one comorbidity, another factor relevant to the prognosis associated with the disease, or any combination thereof.

In various implementations of the present disclosure, expression patterns of the proteins-of-interest can be identified by obtaining a sample 108 from the lesion 104. In various cases, the care provider(s) 106 may surgically remove the sample 108 from the lesion 104 to determine genomic information associated with the lesion 104. For instance, the care provider 106 could surgically remove a tissue biopsy sample 108 from the lesion 104. Tissue biopsies, for example, can include endoscopic biopsies, core needle biopsies, fine-needle aspiration, shave biopsies, punch biopsies, incisional biopsies, excisional biopsies, bone marrow biopsies, surgical biopsies, and the like.

In some cases, the sample 108 includes a liquid biopsy sample. The liquid biopsy sample 108, for instance, includes blood, plasma, cerebrospinal fluid, sputum, stool, urine, lymphatic fluid, saliva, or some other fluid obtained from the body of the subject 102. In some cases, a blood sample is obtained intravenously from the subject 102. The liquid biopsy sample 108, according to various examples, is a plasma sample obtained from the blood of the subject 102. The liquid biopsy sample 108, for instance, can be obtained in a minimally invasive procedure, which could be performed by a medical technician rather than a surgeon.

The sample 108 includes nucleic acid molecules. According to some examples, the nucleic acid molecules include DNA molecules and RNA molecules 110. For instance, the RNA molecules 110 include messenger RNA (mRNA) transcribed from DNA (e.g., genomic DNA (gDNA)) of the subject 102. In some examples, the RNA molecules 110 include at least one of transfer RNA (tRNA), ribosomal RNA (rRNA), or non-coding RNA. Examples of non-coding RNA include microRNA (miRNA), small interfering RNA (siRNA), Piwi-interacting RNA (piRNA), small Cajal body-specific RNA (scaRNA), long intergenic non-coding RNA (lincRNA), circular RNA (circRNA), enhancer RNA (eRNA), natural antisense transcripts (NAT), or the like. Various RNA in the nucleic acid molecules may be indicative of proteins expressed in the cells of the subject 102 and/or the lesion 104.

In some examples, the nucleic acid molecules include RNA molecules 110 that are located in, or extracted from, cells in the sample 108. According to some cases, the RNA molecules 110 are extracted from nuclei and the cells in the sample 108 using mechanical shearing and/or the introduction of a chemical (e.g., a detergent). The RNA molecules 110 may be subsequently isolated from proteins and other cellular materials. In some examples, the RNA molecules 110 indicate a full RNA transcriptome of the subject 102 and/or the lesion 104. In various cases, the nucleic acid molecules include DNA (e.g., gDNA) that indicates an entire genome of the subject 102 and/or the lesion 104. Thus, a genome and/or an RNA transcriptome of the subject 102 and/or the lesion 104 can be determined by sequencing the DNA in the nucleic acid molecules and/or the RNA molecules 110 in the nucleic acid molecules. In various cases, the nucleic acid molecules indicate a whole exome of the subject 102 and/or the lesion 104. According to various implementations, the nucleic acid molecules may include tumor DNA (e.g., DNA from tumor cells), tumor RNA, non-tumor DNA (e.g., DNA from non-tumor cells), or non-tumor RNA.

In various implementations, the sample 108 includes cell-free DNA (cfDNA) and/or cell-free RNA (cfRNA). In examples in which the subject 102 has cancer (e.g., the lesion 104 is a cancerous tumor), the cfRNA, for instance, includes circulating tumor RNA (ctRNA) and/or non-ctRNA. In cases wherein the lesion 104 is a tumor, cancer cells within the lesion 104 will lyse and release the ctRNA into the bloodstream of the subject 102. These cancer cells, for example, include circulating tumor cells (CTCs). Further, other cells additionally shed non-ctRNA into the bloodstream of the subject.

In various cases, the tissue sample 108 is transported to a location that is remote from the subject 102 for further processing. For example, the tissue sample 108 is removed from the subject 102 in a clinical environment (e.g., a hospital) and is then transported to a remote laboratory for further testing and analysis.

The care provider 106 may review the tissue sample using sequencing-based approaches. For instance, the care provider 106 may extract and sequence gDNA from the tissue sample using, in various cases, a sequencing technique (e.g., Sanger sequencing, next-generation sequencing, etc.). In some examples, the care provider 106 may determine, based on the sequenced gDNA, that the subject 102 has heterozygous DNA expression (e.g., expression of both alleles) of a gene for at least one of the proteins-of-interest. In some examples, the care provider 106 may determine, based on the sequenced gDNA, that the subject 102 has loss of DNA heterozygosity (e.g., DNA expression of one allele or no DNA expression) of the gene.

In some instances, the cells within the lesion 104 include one or more copies of a gene for a protein-of-interest that is targeted by a particular therapy. Nevertheless, the treatment(s) may be ineffective if the genes are not expressed for one or more reasons. For instance, loss of DNA heterozygosity of the gene can lead to loss of function of the gene. Loss of function, in various cases, is associated with efficacy of particular therapies (e.g., immunotherapies), disease prognosis, and other disease-related characteristics. Accordingly, determining loss of heterozygosity and/or loss of function of a gene can enable the care provider 106 to provide more accurate information, such as a disease prognosis and/or a therapy predicted to effectively treat a disease of the subject 102, to the subject 102.

However, the genomic expression (e.g., DNA expression) of one or more alleles of a gene may not be representative of true expression of the one or more alleles of the gene. While genomic expression of a homozygous genotype can indicate loss of heterozygosity, homozygosity is not necessarily indicative of loss of function. Furthermore, a gene may become nonfunctional for various reasons other than loss of heterozygosity that cannot be detected by genomic expression. For instance, the gDNA may be indicative of heterozygous DNA expression of a gene, but the RNA expression and/or the protein expression of the gene may indicate loss of heterozygosity. RNA expression and protein expression may differ from DNA expression due to, for instance, epigenetic mechanisms, such as DNA methylation, histone modification, and chromatin structure. Further, the genomic expression may not accurately indicate loss of function, due to, for instance, transcription-related mutations in the RNA molecules 110. In various examples, loss of heterozygosity and loss of function may be incorrectly classified using these techniques. Accordingly, the care provider 106 may not be able to accurately identify true expression of a protein-of-interest associated with a particular therapy in the subject 102 based on the genomic expression of the cells of the lesion 104.

In some examples, the care provider 106 could identify protein expression of the one or more alleles of the gene in the sample 108 using histochemistry and/or immunohistochemistry. For instance, the care provider 106 could surgically remove a tissue biopsy from the lesion 104 and/or review the tissue sample using histochemistry and/or immunohistochemistry. The care provider 106 may apply a tagged antibody configured to bind to a protein corresponding to a particular allele of a gene. However, histopathological approaches may have various drawbacks, such as drawbacks associated with financial cost, time, and accuracy. Further, these techniques may be limited by sensitivity and the ability to detect multiple alleles, including variants thereof.

In various implementations of the present disclosure, true expression of one or more genes of the subject 102 can be determined more accurately, for instance, based on RNA expression of the one or more genes. A sequencer 112 is configured to generate sequence read data 114 indicating the sequences of the RNA molecules 110. The sequencer 112, for instance, includes one or more devices that are configured to generate the sequence read data 114 by processing at least a portion of the sample 108. In some cases, the RNA molecules 110 are extracted from the sample 108. The extraction can be performed by the sequencer 112, by another device, manually (e.g., by a laboratory technician), or any combination thereof. Any appropriate extraction method known to those of ordinary skill in the art can be utilized.

In various cases, the sequencer 112 is configured to perform one or more processes (e.g., chemical reactions) on the RNA molecules 110 in order to prepare the RNA molecules 110 for sequencing. For instance, the sequencer 112 may cause the reverse-transcription of the RNA molecules 110 by applying reverse transcriptase, primers (e.g., oligonucleotide deoxythymidine (oligo dT) primers), and deoxynucleotide triphosphates to the RNA molecules 110, thereby generating complementary DNA (cDNA) molecules. In various implementations, the cDNA molecules include sequences that are complementary to the RNA molecules 110 from the sample 108. In some examples, the sequencer 112 may ligate adapters onto the cDNA molecules and/or amplify the cDNA molecules, such that numerous copies of the ligated cDNA molecules are available for sequencing. Examples of the adapters include, for example, amplification primers, flow cell adapter sequences, substrate adapter sequences, or sample index sequences. The cDNA molecules (e.g., the ligated cDNA molecules) may be amplified by generating multiple copies of the cDNA molecules using one or more techniques such as polymerase chain reaction (PCR), a non-PCR amplification technique, an isothermal amplification technique, linear antisense RNA (aRNA) amplification, or the like. In some cases, the sequencer 112 is configured to perform whole exome sequencing (WES) on the cDNA molecules.

The sequencer 112 may identify the length, position, and identity of the bases in the RNA molecules 110 by sequencing the cDNA molecules (e.g., the amplified and/or ligated cDNA molecules). Based on sequencing the cDNA molecules, the sequencer 112 may determine the sequences of the RNA molecules 110. In various cases, the sequencer 112 is a next-generation sequencer configured to perform next-generation sequencing (NGS) on the cDNA molecules. In various implementations, the sequencer 112 utilizes first-generation sequencing (e.g., Sanger sequencing), second-generation sequencing (e.g., massive parallel sequencing), third-generation sequencing (e.g., nanopore sequencing), or a combination thereof. In some cases, the sequencer 112 is configured to sequence substantially all of the nucleotides of all of the cDNA molecules obtained from the sample 108. In some examples, the sequencer 112 is configured to perform targeted sequencing. For instance, the sequencer 112 may determine whether the RNA molecules 110 contain one or more predetermined sequences at one or more genomic locations based on sequencing the cDNA molecules.

In various cases, the sequencer 112 includes one or more sensors that are configured to detect physical signals (also referred to as “detection signals”) that are indicative of the nucleotide sequences of the cDNA molecules. The sequencer 112 may perform sequencing-by-synthesis. For example, the sequencer 112 may include one or more optical sensors configured to detect optical signals emitted from fluorescently tagged nucleotide triphosphates (NTPs) that are joined together in a synthesized DNA strand using the ligated cDNA molecules as templates. The optical signals detected by the optical sensor(s), for instance, are indicative of the sequences of the cDNA molecules. The sequencer 112 may perform nanopore sequencing. In various cases, the sequencer 112 includes one or more electrical sensors configured to measure an electrical signal (e.g., an electrical current) across a substrate as the ligated cDNA molecules are directed through a nanopore extending through the substrate. The electrical signal over time, in various cases, is indicative of the sequences of the cDNA molecules. The sequencer 112, in various implementations, is configured to generate the sequence read data 114 as digital data based on the analog signals detected by the sensor(s). For instance, the sequencer 112 includes one or more analog to digital converters (ADCs). In various cases, the sequencer 112 includes at least one processor configured to generate the sequence read data 114. Based on determining the sequences of the RNA molecules 110 from the sequences of the cDNA molecules, the sequence read data 114 indicates sequences of RNA present in the sample 108, which may be indicative of the transcriptome of the subject 102 and/or the lesion 104.

In various cases, the sequencer 112 performs sequencing on a subset of the cDNA molecules. For instance, the sequencer 112 may perform targeted sequencing on portions of the cDNA molecules that correspond to one or more predetermined genes and/or loci. In various cases, the genes include HLA-A, HLA-B, HLA-C, HLA-D, HLA-E, HLA-F, HLA-G, HLA-DP, HLA-DPA, HLA-DPA1, HLA-DPA2, HLA-DPB, HLA-DPB1, HLA-DPB2, HLA-DQ, HLA-DQA, HLA-DQA1, HLA-DQA2, HLA-DQB, HLA-DQB1, HLA-DQB2, HLA-DQB3, HLA-DR, HLA-DRA, HLA-DRB, HLA-DRB1, HLA-DRB2, HLA-DRB3, HLA-DRB4, HLA-DRB5, HLA-DM, HLA-DMA, HLA-DMB, HLA-DO, HLA-DOA, or HLA-DOB. In various cases, the genes include one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, B2M, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1,CDK12, CDK4, CDK6, CDK8, CDKN1A, CDKN1B, CDKN2A, CDKN2B, CDKN2C, CEBPA, CHEK1,CHEK2, CIC, CIITA, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERAP1, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1,ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NLRC5, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PSMB1, PSMB2, PSMB8, PSMB9, PSMB10, PSME1, PSME2, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TAP1, TAP2, TAPBP, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB. In some examples, the genes include one or more of POLE, TP53, CTNNNB1, L1CAM, PTEN, ERBB2, PMS2, MSH2, MSH6, MLH1, an estrogen receptor (ER) gene, or a progesterone receptor (PR) gene. Other portions of the genome may be specifically sequenced, such as promoters, hotspots, CpG sites, or other portions of the genome that are not specifically genes but have an impact on genomic expression. The sequencer 112, in some cases, may refrain from sequencing at least a portion of the RNA molecules 110 that do not correspond to the subset.

In various examples, the sequencer 112 is configured to generate the sequence read data 114 indicating sequences of proteins and/or peptides in the sample 108. For instance, the sequencer 112 may extract the proteins and/or the peptides from the sample. The sequencer 112 may digest the proteins using, for instance, an enzyme. In some examples, the sequencer 112 may ionize the digested proteins. For example, the sequencer 112 may use electrospray ionization (ESI), matrix assisted laser desorption ionization (MALDI), atmospheric pressure chemical ionization (APCI), chemical ionization (CI), electron impact (EI), or the like to ionize the digested proteins. Based on ionizing the digested proteins, the sequencer may determine a mass-to-charge ratio of the digested proteins. In various cases, the sequencer 112 may fragment the digested proteins, such as by collision-induced dissociation, electron transfer dissociation, or the like. The sequencer 112 may be configured to determine the sequences of the proteins and/or peptides by analyzing the mass-to-charge ratios of the digested protein and the ions associated with the fragmented proteins. For instance, the sequencer 112 may compare the mass-to-charge ratios of the digested protein and the ions associated with the fragmented proteins to those of known sequences to identify the sequences of the proteins and/or peptides in the sample 108.

In various examples, the sequencer 112 may be configured to image the sample 108 to identify the presence of one or more proteins and/or peptides. For instance, the sequencer 112 may apply a capture agent that is configured to bind a particular protein to the sample 108. Examples of capture agents include antibodies, antibody binding fragments, an aptamer, a peptide, a protein, a small molecule, a nanobody, or the like. The capture agent, in various cases, may be linked to a detectable label, such as a fluorescent probe, a colorimetric dye, a quantum dot, or the like. The sequencer 112 may be configured to detect the detectable label to determine the presence and/or the quantity of the particular protein in the sample. For example, the sequencer 112 may use fluorescence microscopy to detect a fluorescent probe linked to the capture agent in the sample 108.

In some cases, the sequence read data 114 may indicate a whole proteome and/or a whole peptidome of the sample 108. In some cases, as described above in reference to the RNA molecules 110, the sequencer 112 may perform sequencing on a subset of the proteins and/or peptides in the sample 108.

In various implementations, an expression analyzer 116 determines expression of one or more alleles of a gene based on the sequence read data 114. In particular implementations, the expression of an allele is indicative of an RNA expression of the allele and/or a protein expression of the allele. The expression of an allele may be determined based on, for instance, an amount of reads of the sequence read data 114 corresponding to the allele. For instance, in various cases, the amount of reads in the sequence read data 114 corresponding to an allele is assumed to be proportional to the level of expression of that allele by the cells in the lesion 104.

In some implementations, the cells in the lesion 104 include two substantially identical copies of the same allele of the gene (e.g., the cells are homozygous). In some cases, however, the cells in the lesion express multiple alleles of the gene (e.g., the cells are heterozygous). In heterozygous examples, one allele may be expressed at a higher level than the other allele. The allele associated with the higher relative level of expression may be referred to as the “major allele,” and the allele with the lower relative level of expression may be referred to as the “minor allele.”

In various implementations, a relative expression of an allele is compared to a threshold. In particular cases, the expression analyzer 116 determines a ratio representative of the relative expression between different alleles of a gene. For instance, the expression analyzer 116 determines a ratio equal to the number of reads corresponding to the minor allele relative to the total number of reads associated with all alleles of the same gene. In examples with two alleles of the gene, the expression analyzer 116 may determine the total number (e.g., the sum) of reads associated with all alleles of the same gene by adding the number of reads corresponding to the minor allele with the number of reads corresponding to the major allele. In some cases, the expression analyzer 116 determines a ratio equal to the number of reads corresponding to the minor allele relative to the number of reads corresponding to the major allele. Various ratios described herein represent a discrepancy between expression of the alleles of the gene.

According to various cases, the expression analyzer 116 determines whether there is a significant discrepancy in the expression of the alleles of the gene by comparing the ratio to a threshold. For instance, the relative expression of an allele may be compared to a threshold that is in a range of 0.01 to 0.5. In some examples, the relative expression of an allele is compared to a threshold that is in a range of 0.2 to 0.4. In some examples, the relative expression of an allele is compared to a threshold of 0.3. In various cases, the threshold may be determined based on analysis of one or more samples obtained from one or more subjects. In various cases, based on comparing the relative expression of the alleles of the gene to the threshold, the expression analyzer 116 may determine whether the subject 102 has a true heterozygous expression of the gene, rather than a loss of function of at least one allele. For instance, the expression analyzer 116 may determine that the relative expression of a first allele of a gene and the relative expression of a second allele of the gene are above the threshold. Accordingly, the expression analyzer 116 may determine that there is heterozygous expression of the gene. In some examples, the expression analyzer 116 may determine that the relative expression of a first allele of a gene (e.g., the major allele) is above the threshold and that the relative expression of a second allele of the gene (e.g., the minor allele) is below the threshold. Accordingly, the expression analyzer may determine that there is loss of heterozygosity of the gene.

In some cases, the expression analyzer 116 may analyze the DNA expression of the gene. For instance, the expression analyzer 116 may identify true heterozygous expression of the gene based on identifying heterozygous DNA expression of the gene and identifying heterozygous RNA and/or protein expression of the gene. In some examples, the expression analyzer 116 may identify homozygous expression of the gene based on identifying homozygous DNA expression of the gene. In various cases, the expression analyzer 116 may confirm that there is true homozygous expression of the gene by identifying homozygous RNA and/or protein expression of the gene. In some examples, the expression analyzer 116 may identify true homozygous expression of the gene based on identifying heterozygous DNA expression of the gene and identifying homozygous RNA and/or protein expression of the gene. Accordingly, the expression analyzer 116 can more accurately determine the true expression as well as the function or loss of function of the gene by analyzing the RNA and/or protein expression rather than the DNA expression alone.

In various implementations, the expression analyzer 116 is configured to determine the allelic expression of more than one gene. In some examples, the expression analyzer 116 may use different threshold for each gene. For instance, the expression analyzer 116 may determine the allelic expression of one or more HLA class I genes (e.g., HLA-A, HLA-B, HLA-C, etc.) and the allelic expression of one or more HLA class II genes (e.g., HLA-DP, HLA-DQ, HLA-DR, etc.).

In some examples, the expression analyzer 116 is configured to determine one or more expression indicators 118 based on the RNA and/or protein expression of the alleles of the one or more genes in the sample 108. The expression indicator(s) 118, in various cases, are indicative of the expression of the allele of the one or more genes in the sample 108. For instance, the expression indicator(s) 118 may indicate the expression (e.g., the number of reads) of each allele of the one or more genes and/or the relative expression of each allele of the one or more genes. In some examples, he expression indicator(s) 118 indicate whether the relative expression of each allele of the one or more genes is greater than the threshold corresponding to the gene. For instance, the expression indicator(s) 118 may indicate whether the sample 108 is associated with heterozygous expression or loss of heterozygosity of the one or more genes.

In various implementations, the expression indicator(s) 118 indicate a predicted (e.g., suggested) effective therapy to treat the lesion 104 of the subject 102. For instance, the expression indicator(s) 118 may indicate a likelihood that a particular therapy will effectively treat the lesion 104 of the subject 102. In some implementations, the expression indicator(s) 118 indicate a predicted immunotherapy to treat the lesion 104 of the subject 102 based on, for instance, the RNA and/or protein expression of the alleles of one or more immune-related genes and/or cancer-related genes of the subject 102. For instance, the expression analyzer 116 may determine the predicted immunotherapy based on the RNA expression of the alleles of one or more immunotherapy targets (e.g., PD-1/PDL-1) and/or one or more proteins involved in the therapeutic response. In various examples, the expression indicator(s) 118 indicate a predicted immunotherapy to treat the lesion 104 of the subject 102 based on the RNA and/or protein expression of the alleles of one or more HLA genes of the subject 102. For instance, the expression indicator(s) 118 may indicate that tebentafusp-tebn is predicted to effectively treat the lesion 104 of the subject 102 based at least in part on RNA expression of HLA-A* 02:01 in the subject 102. In various examples, the expression indicator(s) 118 indicate that afamitresgene autoleucel is predicted to effectively treat the lesion 104 of the subject 102 based on RNA expression of HLA-A* 02:01, HLA-A*02:02, HLA-A*02:03, or HLA-A*02:06 in the subject 102. The expression indicator(s) 118 may indicate that the predicted immunotherapy is approved for treatment of the cancer of the subject, such as tebentafusp-tebn for treatment of uveal melanoma for afamitresgene autoleucel for treatment of synovial sarcoma. In some cases, the expression indicator(s) 118 may indicate a predicted effective target of the immunotherapy. For instance, the expression indicator(s) 118 may indicate that a T-cell receptor therapy targeted to the MAGE-A3 protein may effectively treat the lesion 104 of the subject 102.

In some examples, the expression indicator(s) 118 may indicate a predicted cancer type and/or cancer subtype associated with the lesion 104. In various implementations, the expression indicator(s) 118 are indicative of at least one predicted cell type of the lesion 104 of the subject 102 and/or a predicted tumor progression of the lesion 104. The expression indicator(s) 118 may indicate, in some cases, a predicted genomic and/or phenotypic cell type of the lesion 104. For instance, the predicted tumor progression may indicate a rate of growth of the lesion 104. In some examples, the predicted tumor progression indicates a rate of growth of one or more predicted cell types of the lesion 104. In various cases, the predicted tumor progression indicates a predicted metastasis profile of the subject 102. The predicted metastasis profile may indicate a time (e.g., a date, a time range) when the lesion 104 will metastasize. In some examples, the expression indicator(s) 118 include a likelihood that the lesion 104 will metastasize (e.g., to the lymph nodes, or to a particular organ) by a given time or an indication that there is greater than a threshold (e.g., 80%) likelihood that the lesion 104 will metastasize by the given time. In various cases, the expression indicator(s) 118 may indicate a prognosis of the subject 102. The prognosis of the subject 102 may indicate a predicted survivability of the subject 102.

In various examples, the expression indicator(s) 118 include a predicted condition (e.g., disease) of the subject 102; a predicted disease subtype of the subject 102; a predicted survivability of the subject 102; one or more predicted symptoms of the subject 102; a dosage of one or more therapeutic agents (e.g., biologics, chemotherapeutic agents, etc.) predicted to treat the condition of the subject 102, a predicted stage of the predicted disease of the subject 102; a predicted grade of the predicted disease of the subject 102; a predicted activity level of the subject 102 (e.g., a predicted Eastern Cooperative Oncology Group (ECOG) performance status of the subject 102); a predicted body mass index (BMI) of the subject 102; a predicted smoking history of the subject 102; a predicted breast density of the subject 102; a clinical trial that the subject 102 is predicted to qualify (e.g., be eligible) for; or a characteristic of the predicted disease of the subject. For instance, the expression indicator(s) 118 may indicate that the subject 102 is likely to qualify for a clinical trial based on an age, a gender, a disease stage, and previous treatments of the subject 102.

In some implementations, the expression analyzer 116 is unable to conclusively categorize the allelic expression of one or more genes of the subject 102. In various cases, the expression indicator(s) 118 may include an indication that that the categorization of the allelic expression of the one or more genes is inconclusive.

In various implementations, the expression analyzer 116 determines the expression indicator(s) 118 based other characteristics of the subject 102. In various implementations, the characteristics include the sequences of the RNA molecules 110 indicated in the sequence read data 114. In some examples, the characteristics include the sequences of the proteins and/or peptides indicated in the sequence read data 114. In various examples, the characteristics may include copy number variation associated with one or more of the RNA molecules 110.

In various examples, the characteristics are based on fragments in the RNA molecules 110, and are therefore referred to as “fragmentomic features.” Examples of fragmentomic features include endpoint positions of the fragments in a reference genome (e.g., right endpoints, left endpoints, etc.), endpoint counts at positions within the reference genome (e.g., right endpoint counts, left endpoint counts, etc.), fragment lengths, end motifs, relative read depths of the fragments, the presence of one or more variants in the fragments, or any combination thereof.

In various implementations, the expression analyzer 116 may include a predictive model that is configured to generate the expression indicator(s) 118 based on the relative expression of the alleles of one or more genes. The predictive model, for example, may include one or more mathematical and/or computer-based models that are configured to predict the expression indicator(s) 118. For instance, the predictive model may include a regression model, threshold rule, confidence interval, or other type of statistical model capable of categorizing the expression indicator(s) 118. In various cases, the predictive model includes at least one classifier configured to generate the expression indicator(s) 118.

In various implementations, the predictive model includes at least one trained ML model configured to output the expression indicator(s) 118. For example, parameters of the ML model(s) may have been previously optimized based on training data including features of individuals within a population omitting the subject 102. For instance, the ML model(s) was trained using an unsupervised or semi-supervised learning technique to identify attributes of the training data that are indicative of the expression indicator(s) 118, wherein the parameters were optimized to categorize (e.g., cluster) the features of the population. In some cases, the ML model(s) was trained using a supervised learning technique, wherein the training data further included ground truth conditions (e.g., allelic expression, health-related conditions, etc.) of the individuals in the population, such that the parameters were optimized to minimize a loss between predicted health-related conditions generated by the ML model(s) based on the features of the population and the ground truth conditions of the cancers experienced by the individuals in the population. To increase training robustness, the population represented by the training data may include individuals without the particular allelic expression, individuals without the health-related condition, individuals without cancer, as well as individuals with a variety of types of presentations of the allelic expression and/or health-related condition. Various types of ML models can be included in the predictive model, such as a neural network, a nearest-neighbor model, a regression analysis model, a clustering model, a principal component analysis model, a gradient boosting model, a random forest, or any combination thereof. In some cases, the predictive model includes a hybrid model, that includes multiple types of ML models. For instance, the predictive model may include a CNN and a clustering model.

A report generator 120 is configured to generate a report 122 based, at least in part, on the expression indicator(s) 118. The report 122, for example, includes consumable data that can inform the care provider 106 about the predicted condition of the subject 102.

In various implementations, the report 122 may indicate the results of additional analyses, such as the results of a histological study, whole transcriptome sequencing, a targeted RNA sequencing test, a long-read RNA sequencing test, cfRNA sequencing, a comprehensive genomic profiling test, whole exome sequencing, whole genome sequencing, a cancer hotspot panel test, a DNA methylation test, a TMB test, a DNA fragmentation test, an RNA fragmentation test, a microsatellite instability (MSI) test, or a viral status test. The performance of such tests is within the ordinary skill of the art, with additional detail provided elsewhere herein. The report 122, for example, may include a genomic profile of the subject 102 based on various combinations of the above analyses and tests.

In some implementations, the report 122 indicates that a follow-up test of the subject 102 is indicated. For instance, in response to determining that the categorization of the allelic expression of the subject 102 is inconclusive, the report generator 120 may generate the report 122 to indicate that one or more additional tests, such as a physical exam, a histological study, or a nucleic acid sequencing-based test (e.g., genome sequencing, exome sequencing, additional DNA sequencing, RNA sequencing, transcriptome sequencing, etc.) should be performed in order to accurately identify the allelic expression and/or the health-related condition of the subject 102. In some examples, the one or more addition tests may include diagnostic imaging, such as magnetic resonance imaging, computed tomography scan, ultrasound, X-ray, mammogram, positron emission tomography, bone scintigraphy, myelography, virtual colonoscopy, echocardiography, radiography, nuclear medicine, fluoroscopy, or single-photon emission computed tomography.

In various cases, the report 122 is output to a clinical device 130. For example, the report generator 120 transmits the report 122 to the clinical device 130. In various implementations, the clinical device 130 is a computing device that is operated by, owned by, or otherwise associated with the care provider 106. For instance, the clinical device 130 may be a desktop computer, a laptop computer, a smart phone, or some other computing device associated with the care provider 106. The clinical device 130, in various cases, outputs the report 122 to the care provider 106. In some cases, the clinical device 130 includes a display (e.g., a screen) that visually presents the report 122. In various cases, the clinical device 130 includes a speaker that outputs a sound indicative of the report 122. The clinical device 130, in various cases, may output the information in the report 122 using one or more output mechanisms or devices.

The care provider 106 may review the report 122 by interacting with the clinical device 130. The report 122, in various cases, may enhance the clinical decision-making of the care provider 106. For instance, the care provider 106 may determine a therapy for the subject 102 based on the report 122, such as drug therapy, radiation therapy, targeted therapy, vaccine therapy, stem cell transplantation, blood transfusion, physical therapy, psychiatric therapy, or surgery. The care provider 106 may prepare and/or administer a therapy to the subject 102 based on the report 122. According to various implementations, the care provider 106 may initiate the therapy and/or refer the subject 102 to another care provider to receive the therapy. In various cases, if the predicted condition of the subject 102 is a disease (e.g., cancer), the care provider 106 may prescribe, recommend, or administer an agent in order to treat the disease the subject 102.

In various implementations, the care provider 106 may develop a diagnosis and/or prognosis of the subject 102 based on the report 122. In various implementations, the care provider 106 may communicate information in the report 122 to the subject 102.

FIG. 1 illustrates various elements that can be embodied in one or more computing devices. For example, at least a portion of the functions of the sequencer 112, the expression analyzer 116, the report generator 120, or any combination thereof are performed by one or more processors in at least one computing device. Examples of computing devices include server computers, desktop computers, laptop computers, tablet computers, mobile phones, wearable devices, Internet of Things (IoT) devices, and the like. In various cases, instructions for performing at least a portion of the functions of these elements are stored in memory and/or in a non-transitory computer readable medium. The instructions, for instance, are executed by the processor(s).

FIG. 1 also illustrates various types of data. For example, the sequence read data 114, the expression indicator(s) 118, the report 122, or any combination thereof, includes data. The various types of data illustrated in FIG. 1 may be stored, such as in memory or in non-transitory computer readable media. In various implementations, at least a portion of the data is transmitted or otherwise output by one or more computing devices. For example, a computing device may transmit one or more communication signals to another computing device, wherein the communication signal(s) encode at least a portion of the data. Examples of communication signals include electromagnetic signals, optical signals, ultrasonic signals, optical signals, and electrical signals. For example, communication signals can be transmitted wirelessly and/or in a wired fashion. The communication signals, for instance, are transmitted over one or more wireless channels and/or one or more wired channels (e.g., optical cabling, electrical cabling, etc.). In various cases, the communication signal(s) are transmitted over one or more communication networks. A communication network, for instance, may be defined according to one or more physical channels, such as one or more frequency spectra. In some cases, a communication network is defined according to one or more communication protocols and/or standards. Examples of communication networks include fiber optic networks, Institute of Electrical and Electronics Engineers (IEEE) networks (e.g., WI-FI™ networks, WiMAX networks, BLUETOOTH™ networks, etc.), cellular networks (e.g., a 3^{rd} Generation Partnership Project (3GPP) radio network, such as a Long Term Evolution (LTE) network, a New Radio (NR) network; or a cellular core network such as a 3^{rd} Generation (3G) core, a 4^{th} Generation (4G) core, a 5^{th} Generation (5G) core, etc.), ultrasonic networks, and the like. In some cases, the data is broadcasted from one device to multiple other devices. In some cases, the data is unicasted from one device to another device. For instance, various forms of data described herein may be transmitted via a peer-to-peer (P2P) connection.

A particular example will now be described with reference to FIG. 1. In this example, the subject 102 presents to a clinical environment for an assessment (e.g., a follow-up visit) of a lung cancer of the subject 102. The care provider(s) 106 may initiate and/or identify results of medical imaging (e.g., computed tomography (CT) imaging) on the subject 102. In some examples, the care provider(s) 106 may determine a location of the lesion 104 on a lung of the subject 102. The care provider(s) 106 may obtain the sample 108, for instance, by performing a needle biopsy procedure on the lesion 104.

The sequencer 112 may generate sequence read data 114 based on RNA molecules 110 within the tissue sample 108 of the subject 102. For example, the sequence read data 114 may represent RNA molecules associated with one or more genes associated with efficacy of a particular therapy to treat the color cancer of the subject 102. For instance, the sequence read data 114 may represent RNA molecules 110 associated with one or more human leukocyte antigen (HLA) genes. Examples of HLA genes include HLA-A, HLA-B, HLA-C, HLA-D, HLA-E, HLA-F, HLA-G, HLA-DP, HLA-DPA, HLA-DPA1, HLA-DPA2, HLA-DPB, HLA-DPB1, HLA-DPB2, HLA-DQ, HLA-DQA, HLA-DQA1, HLA-DQA2, HLA-DQB, HLA-DQB1, HLA-DQB2, HLA-DQB3, HLA-DR, HLA-DRA, HLA-DRB, HLA-DRB1, HLA-DRB2, HLA-DRB3, HLA-DRB4, HLA-DRB5, HLA-DM, HLA-DMA, HLA-DMB, HLA-DO, HLA-DOA, or HLA-DOB. In various examples, the expression analyzer 116 may determine the relative RNA expression of one or more alleles of the one or more HLA genes in the sample 108. For instance, the relative RNA expression of an allele of a gene may be indicative of a ratio of the RNA expression of the allele of the gene to the RNA expression of all alleles of the gene. The expression analyzer 116 may compare the ratio of the one or more alleles of the one or more HLA genes to a threshold. The expression analyzer 116 may determine the expression indicator(s) 118.

In some examples, the expression analyzer 116 may determine that the relative RNA expression of a first allele of HLA-B is above the threshold and that the relative RNA expression of a second allele of HLA-B is lower than the threshold. Accordingly, the expression indicator(s) 118 may indicate that the lesion 104 may be associated with homozygous expression of HLA-B. The expression indicator(s) 118 may indicate that an immune checkpoint inhibitor (e.g., a PD-1 inhibitor) is predicted to not effectively treat the lesion 104 based on the relative expressions of the first allele and the second allele of HLA-B.

In some examples, the expression analyzer 116 may determine that the relative RNA expression of a first allele of HLA-DP is above the threshold and that the relative RNA expression of a second allele of HLA-DP is above the threshold. Accordingly, the expression indicator(s) 118 may indicate that the lesion 104 may be associated with heterozygous expression of HLA-DP. The expression indicator(s) 118 may indicate that a T-cell receptor therapy (e.g., chimeric antigen receptor T-cell (CAR T-cell) therapy) is predicted to effectively treat the lesion 104 based on the relative expressions of the first allele and the second allele of HLA-DP. In various cases, the expression indicator(s) 118 may indicate a particular target for the second therapy (e.g., epidermal growth factor receptor (EGFR)-targeted CAR T-cell therapy).

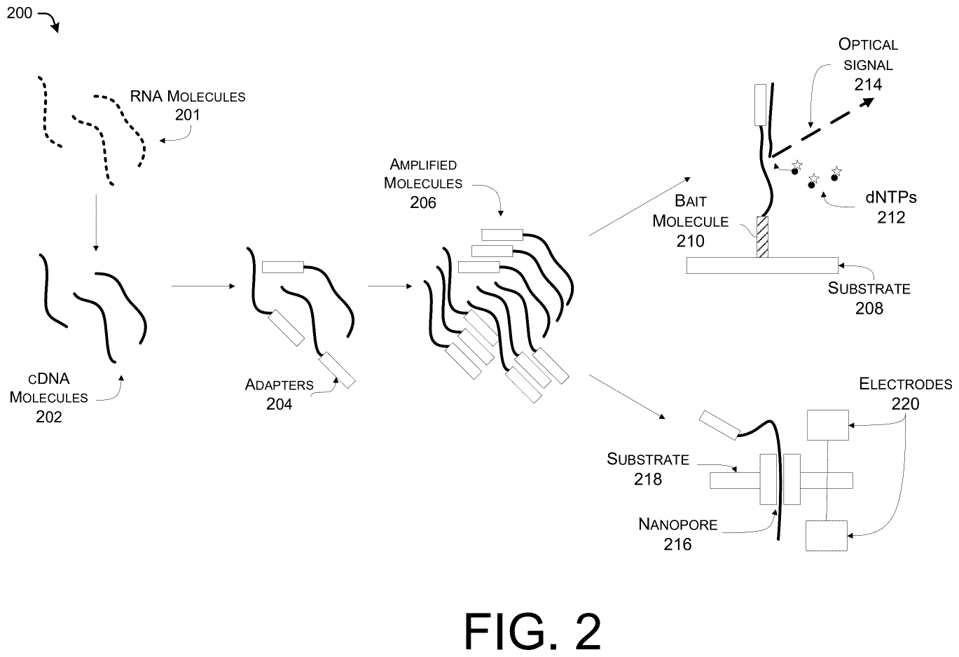

FIG. 2 illustrates an example environment 200 for sequencing various RNA molecules 201. In various implementations, the RNA molecules 201 include mRNA. In some examples, the RNA molecules 201 include transfer RNA (tRNA), ribosomal RNA (rRNA), non-coding RNA, or the like. In various implementations, the RNA molecules 201 may include circulating tumor RNA (ctRNA) and/or cell-free RNA (cfRNA) from non-cancerous cells. The RNA molecules 201, in various cases, are extracted from a sample, such as a biological sample obtained from a subject. In some implementations, RNA molecules 201 are extracted from a tissue biopsy sample collected from a tumor of the subject.

In various implementations, the RNA molecules 201 are reverse-transcribed into complementary DNA (cDNA) molecules 202. The cDNA molecules 202, in various cases, are ligated with adapters 204. For examples, the adapters 204 are hybridized to the cDNA molecules 202. The adapters 204, for example, include additional nucleic acid molecules. In various implementations, the adapters 204 have a shorter length than the cDNA molecules 202 being sequenced. For instance, the adapters 204 include amplification primers, flow cell adapter sequences, substrate adapter sequences, or sample index sequences. Although FIG. 2 illustrates adapters 204 being ligated to one end of each of the cDNA molecules 202, implementations are not so limited. For example, the adapters 204 may be ligated to both ends of each of the cDNA molecules 202.

In various examples, the cDNA molecules 202 ligated with the adapters 204 are amplified in order to generate amplified molecules 206. Various amplification techniques can be performed. For instance, the amplified molecules 206 are generated using PCR, a non-PCR amplification technique, an isothermal amplification technique, or any combination thereof.

Amplified molecules 206 may be captured by bait molecules 210 and sequenced. In various cases, the bait molecules 210 include first bait molecules associated with a first allele of a gene and second bait molecules associated with a second allele of the gene. In various examples, the first bait molecules include multiple first bait molecules configured to capture amplified molecules 206 associated with variants of the first allele of the gene. In various examples, the second bait molecules include multiple second bait molecules configured to capture amplified molecules 206 associated with variants of the second allele of the gene. In some cases, the bait molecules 210 target one or more exonic regions (e.g., exons) of one or more alleles of the gene. In various implementations, the bait molecules 210 are configured to target all exons of each allele of the gene.

In some implementations, the amplified molecules 206 are sequenced via sequencing-by-synthesis. In various cases, fluorescently tagged deoxyribonucleotide triphosphates (dNTP) 212 are utilized to synthesize a strand that is complementary to DNA strands bound to the substrate 208. When a dNTP 212 is added to the strand (e.g., by an enzyme), the dNTP 212 emits an optical signal 214. In various implementations, the frequency of the optical signal 214 is dependent on the type of dNTP 212 from which the optical signal 214 is emitted. By detecting the optical signals 214 as the strand is being synthesized, the sequence of the original cDNA molecules 202 can be derived.

In some implementations, the amplified molecules 206 are sequenced via nanopore sequencing. For instance, the amplified molecules 206 are directed through a nanopore 216 extending through a substrate 218. In various cases, the amplified molecules 206 are negatively charged, such that they can be directed through the nanopore 216 by imposing an electrical field across the substrate 218. In various cases, the amplified molecules 206 and the nanopore 216 are in the presence of a charged solution. Thus, charged solutes traveling through the nanopore 216 can be monitored by reviewing an electrical signal (e.g., a current) sensed between electrodes 220 on either side of the substrate 218. As an amplified molecule 206 is directed through the nanopore 216, the individual bases within the amplified molecule 206 will block the nanopore 216, which may decrease the amount of charged solutes traveling through the nanopore 216 and consequently, the magnitude of the electrical signal detected by the electrodes 220. Each of the four types of bases within the amplified molecules 206, may block the nanopore 216 to a different extent. Therefore, the sequence of the nucleic acid molecules 202 can be derived by analyzing the measured electrical signal with respect to time as the amplified molecules 206 are directed through the nanopore 216.

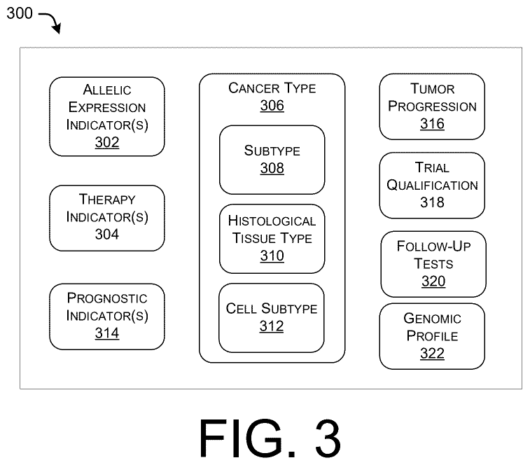

FIG. 3 illustrates an example report 300 summarizing predicted categories of a cancer of a subject. In various cases, the report 300 is the report 122 described above with reference to FIG. 1. The report 300, for instance, may be displayed to a patient and/or care provider. In some cases, the report 300 is generated based on features of a sample (e.g., a tissue biopsy sample) obtained from the subject.

In various cases, the report 300 includes one or more allelic expression indicators 302, such as at least one of an RNA expression, a protein expression, or a DNA expression, associated with one or more genes of the subject. The allelic expression indicator(s) 302 may indicate an expression level (e.g., a number of reads, a signal, etc.) associated with one or more alleles of the one of more genes of the subject. The allelic expression indicator(s) 302 may indicate a relative expression of one or more alleles of the one of more genes of the subject. In various cases, the allelic expression indicator(s) 302 indicate whether the relative expression of one or more alleles of the one of more genes of the subject is greater than a threshold or lower than a threshold. In some examples, the allelic expression indicator(s) 302 indicates a heterozygosity or a homozygosity associated with the one or more genes of the subject.

In various cases, the report 300 includes one or more therapy indicators 304. For instance, the therapy indicator(s) 304 convey whether the cancer is predicted to be resistant to one or more predetermined therapies and/or whether the cancer is predicted to be responsive to one or more predetermined therapies.

The report 300 includes a cancer type 306. The cancer type 306 may be indicative of a subtype 308 of the cancer. In various examples, the cancer type 306 indicates a histological tissue type 310, a cell subtype 312, or any combination, of the cancer.

In some examples, the report 300 includes one or more prognostic indicators 314. The prognostic indicator(s) 314, for instance, indicate a prognosis of the subject in view of the categorized cancer. For example, the prognostic indicator(s) 314 may indicate a survivability, a recoverability, a quality of life indicator, or other information indicative of the prognosis of the subject.

The report 300, in various implementations, includes a tumor progression 316 of the subject. The tumor progression 316, for instance, indicates a metastasis profile of the subject indicative of a likelihood that the cancer will metastasize (e.g., at a particular point in time), one or more tissues in which the cancer is predicted to metastasize, or the like. In some examples, the tumor progression 316 is indicative of a predicted rate of growth of a tumor of the subject and/or a predicted rate of growth associated with the predicted cell subtypes of the tumor of the subject.

The report 300 may include a trial qualification 318 of the subject. The trial qualification 318, for instance, indicates whether the subject is predicted to qualify for a predetermined clinical trial.

In various cases, the report 300 includes recommended follow-up tests 320. For example, the report 300 may include a recommendation to perform whole genome sequencing on the subject, particularly in cases if the cancer cannot be categorized above a threshold certainty.

The report 300 may include a genomic profile 322 of the subject. In various cases, the genomic profile 322 includes or is generated based on the results of one or more nucleic acid sequencing-based analyses of the subject.

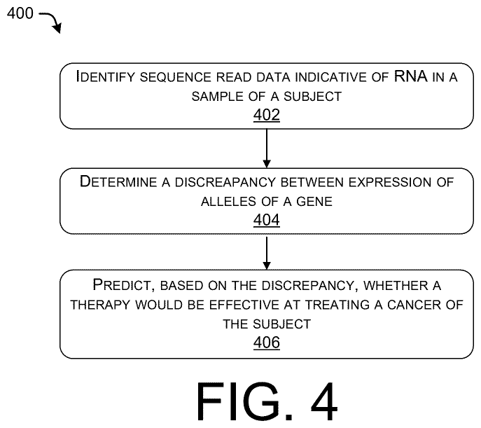

FIG. 4 illustrates an example process 400 for identifying a predicted effective treatment based on an allelic expression of a subject. In various implementations, the process 400 is performed by an entity including at least one processor, at least one computing device, a medical device, a device configured to collect a tissue sample, the sequencer 112, the expression analyzer 116, the report generator 120, the clinical device 130, or any combination thereof.

At 402, the entity identifies sequence read data indicative of RNA in a sample of a subject. For instance, the entity receives a plurality of nucleic acid molecules in a sample of the subject. The sample may include a tissue sample or liquid sample (e.g., a blood sample, a urine sample, a saliva sample, etc.). In some examples, the tissue sample is obtained from a tumor of the subject. The nucleic acid molecules, for instance, include RNA molecules from the sample. The RNA molecules may be reverse transcribed into cDNA molecules. One or more adapters are ligated onto at least some of the cDNA molecules. The ligated cDNA molecules are amplified and captured. In various cases, all or a subset of the captured cDNA molecules are sequenced to obtain a plurality of sequence reads that represent the sequenced amplified cDNA molecules, thereby generating the sequence read data. In various examples, the entity received a plurality of proteins and/or peptides in the sample of the subject. The entity may extract the proteins and/or peptides from the sample and digest the extracted proteins and/or peptides. In some examples, the entity ionizes the digested proteins and/or peptides and determines a mass-to-charge ratio of the ionized proteins and/or peptides. In various cases, the entity fragments the digested proteins and/or peptides. The entity may determine the sequence read data based on analyzing the mass-to-charge ratio of the digested proteins and the ions associated with the fragmented proteins.

At 404, the entity determines a discrepancy between expression of alleles of a gene. The expression of the alleles may include RNA expression and/or protein expression. In various examples, the entity compared a relative expression of the alleles of the gene. For instance, the entity may determine a ratio of the expression of a particular allele of the gene to the expression of all of the alleles of the gene. In various cases, the entity compared the expression of the alleles of the gene to a threshold. In some examples, the entity may determine a heterozygous expression or a loss of heterozygosity of the gene. For instance, the entity may determine that the expression of a first allele of the gene is greater than the threshold and that the expression of a second allele of the gene is lower than the threshold. Accordingly, the entity may determine a loss of heterozygosity associated with the gene. In various examples, the entity may determine the discrepancy between expression of alleles of multiple genes.

At 406, the entity predicts, based on the discrepancy between the expression of the alleles of the gene, whether a therapy would be effective at treating a cancer of the subject. For instance, the entity may determine that a lesion (e.g., a cancerous tumor) of the subject would not respond a particular therapy based on identifying loss of heterozygosity associated with the gene. In various cases, the entity may determine that a different therapy would delay progression of a cancer of the subject. In some examples, the entity may predict a treatment efficacy based on the sequences associated with the alleles of the gene. For instance, the entity may identify a mutation in an expressed allele of the gene that may confer resistance to a particular therapy. In various cases, the entity may predict various health-related conditions, such as a predicted survivability of the subject, a predicted tumor progression of the subject, a predicted metastasis profile of the subject, a predicted cell type of a tumor of the subject, or the like. In some cases, the entity utilized an ML-based classifier to generate a classification of the predicted treatment efficacy based on pre-classified data associated with individuals who do or do not have particular allelic expression of the gene. The ML-based classifier, for instance, is pre-trained based on data obtained from a population of individuals that omits the subject. In some cases, the classifier includes at least one of an artificial neural network (ANN), a logistic regression model, a decision tree, a k-nearest neighbor (KNN) model, a support vector machine (SVM), or a naïve Bayes classifier.

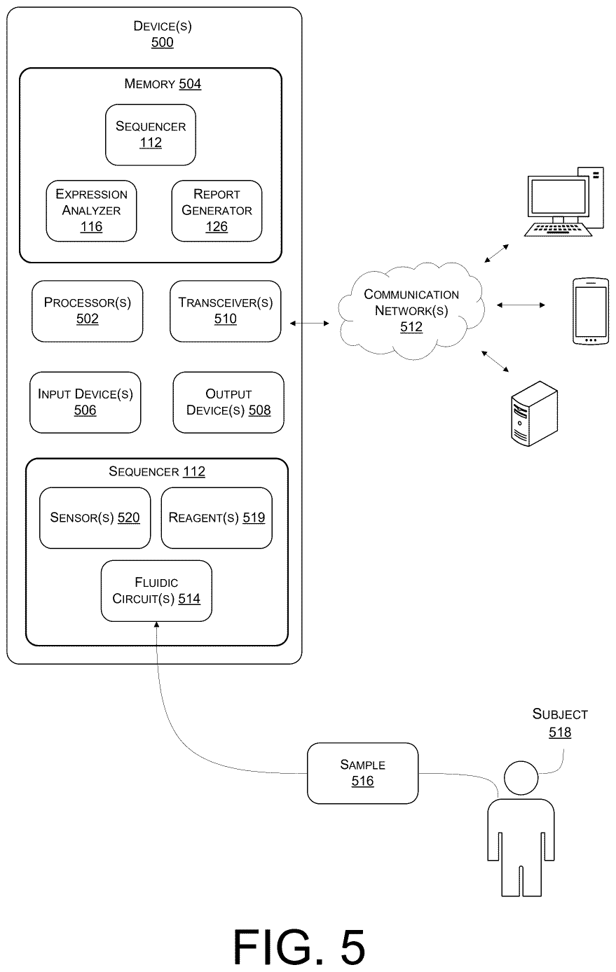

FIG. 5 illustrates one or more devices 500 configured to perform various operations described herein. The device(s) 500 include one or more processor(s) 502. In some implementations, the processor(s) 502 includes a central processing unit (CPU), a graphics processing unit (GPU), both CPU and GPU, or other processing unit or component known in the art.

The processor(s) 502 is operably connected to memory 504. In various implementations, the memory 504 is volatile (such as random access memory (RAM)), non-volatile (such as read only memory (ROM), flash memory, etc.) or some combination of the two. The memory 504 stores instructions that, when executed by the processor(s) 502, causes the processor(s) 502 to perform various operations. In various examples, the memory 504 stores methods, threads, processes, applications, objects, modules, any other sort of executable instruction, or a combination thereof. In some cases, the memory 504 stores files, databases, or a combination thereof. In some examples, the memory 504 includes, but is not limited to, RAM, ROM, electrically erasable programmable read-only memory (EEPROM), flash memory, or any other memory technology. In some examples, the memory 504 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 502. For instance, the memory 504 stores instructions that, when executed by the processor(s) 502, causes the processor(s) 502 to perform operations of the sequencer 112, the expression analyzer 116, and the report generator 120.

The processor(s) 502 is operably connected to one or more input devices 506 and one or more output devices 508. Collectively, the input device(s) 506 and the output device(s) 508 function as an interface between at least one user and the device(s) 500. The input device(s) 506 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 508 includes at least one of a display, a speaker, a haptic output device, a printer, or any combination thereof. In various examples, the processor(s) 502 causes a display among the input device(s) 506 to visually output various data described herein. In some implementations, the input device(s) 506 includes one or more touch sensors, the output device(s) 508 includes a display screen, and the touch sensor(s) are integrated with the display screen.

In various implementations, the processor(s) 502 is operably connected to one or more transceivers 510 that transmit and/or receive data over one or more communication networks 512. For example, the transceiver(s) 510 includes a network interface card (NIC), a network adapter, a local area network (LAN) adapter, or a physical, virtual, or logical address to connect to the various external devices and/or systems. In various examples, the transceiver(s) 510 includes any sort of wireless transceivers capable of engaging in wireless communication (e.g., radio frequency (RF) communication). For example, the communication network(s) 512 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 510 includes other wireless modems, such as a modem for engaging in WI-FI®, WIGIG®, WIMAX®, BLUETOOTH®, or infrared communication over the communication network(s) 512

The device(s) 500 may further include the sequencer 112. In various implementations, the sequencer 112 includes one or more fluidic circuits 514 configured to receive a sample 516 derived from a subject 518. The sequencer 112, in various cases, may be configured to generate data indicative of one or more sequences of nucleic acid molecules (e.g., DNA and/or RNA) present in the sample 516. In various cases, the sequencer 112 introduces one or more reagents 519 to the fluidic circuit(s) 514 in order to prepare for and perform sequencing of the nucleic acid molecules. Further, the sequencer 112 may include one or more sensors 520 configured to measure or otherwise detect detection signals from the fluidic circuit(s) 514, which may be indicative of the sequences of the nucleic acid molecules. According to various implementations, the sensor(s) 520 may further include one or more ADCs. The sequencer 112, in various cases, outputs sequence read data to the processor(s) 502 for additional processing.

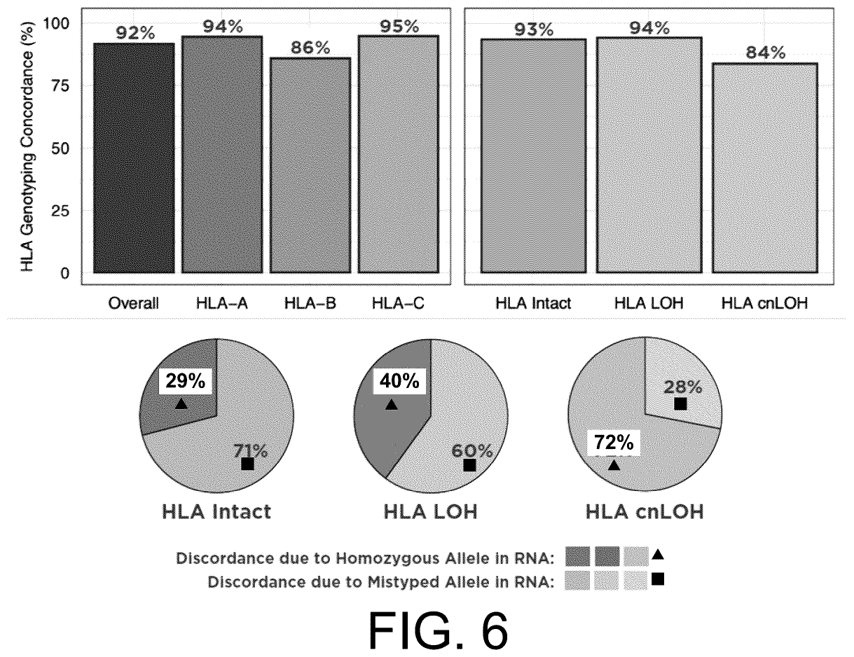

FIG. 6 illustrates HLA genotyping concordance between DNA and RNA samples. Top left, the concordance of all HLA class I alleles as genotyped by DNA and RNA in the overall cohort (n=3552) as well as within each gene (HLA-A, HLA-B, and HLA-C, n=1184 for each). Top right, the HLA genotyping concordance amongst samples that were estimated to be HLA LOH negative (HLA Intact, n=2814) vs HLA LOH positive (HLA LOH, n=84, or HLA cnLOH, n=654). Bottom, for samples that had discordant HLA genotyping results, the percent of HLA alleles (as typed by DNA) that were either mistyped (i.e. called a different allele) by RNA or typed as homozygous by RNA.

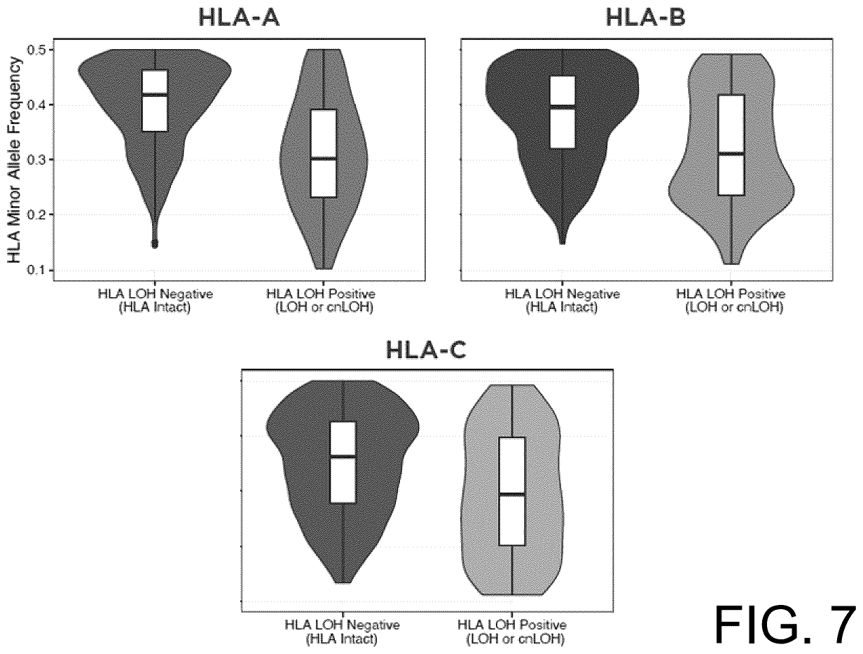

FIG. 7 illustrates that lower median HLA minor allele frequency (MAF) is seen in samples that are HLA LOH positive. Amongst genes that were genotyped as germline heterozygous in both DNA and RNA, the MAF is shown stratified by whether the sample was HLA LOH Negative (HLA Intact) vs HLA LOH Positive (LOH or cnLOH). HLA-A HLA LOH Negative n=400, HLA-A HLA LOH Positive n=69, HLA-B HLA LOH Negative n=405, HLA-B HLA LOH Positive n=85, HLA-C HLA LOH Negative n=412, HLA-C HLA LOH Positive n=79.

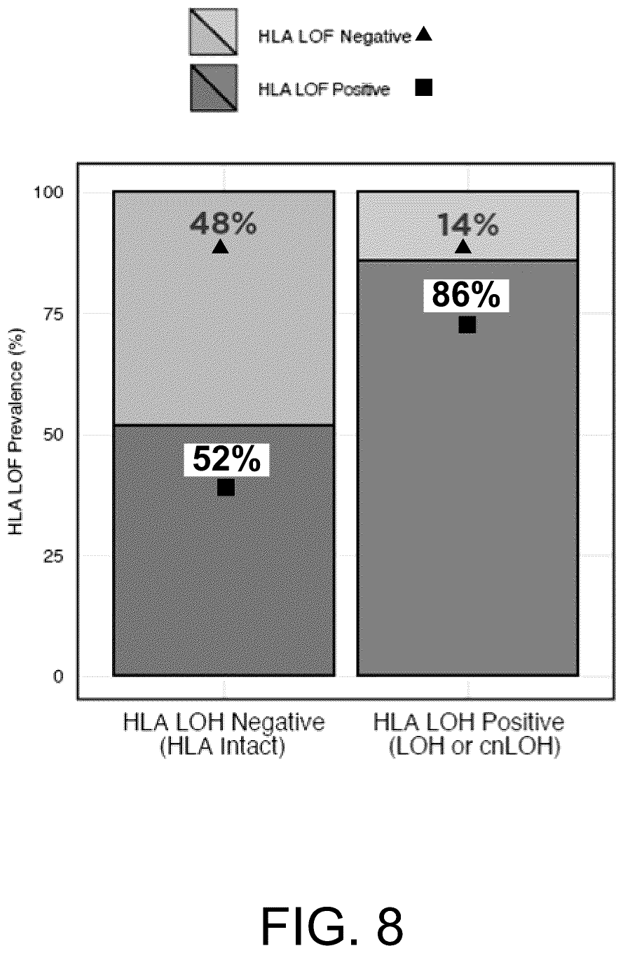

FIG. 8 illustrates prevalence of HLA loss of function (LOF). Prevalence of HLA LOF in samples that have at least one germline heterozygous HLA class I allele. Of samples that were HLA LOH Negative (HLA Intact, n=461), 52% were estimated to have HLA LOF. Of samples that were HLA LOH Positive (LOH or cnLOH, n=119), 86% were estimated to have HLA LOF. In this Example, a sample was categorized as having HLA LOF if at least one HLA class I gene had a minor allele frequency (MAF) below 0.3 or, after being typed as germline heterozygous by DNA, was genotyped as homozygous by RNA.

### Example Clauses

1. A method, including: providing a plurality of RNA molecules obtained from a sample from a subject; generating a plurality of nucleic acid molecules by reverse transcribing the RNA molecules; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, all or a subset of the captured amplified nucleic acid molecules to obtain a plurality of sequence reads that represent the sequenced amplified nucleic acid molecules thereby generating sequence read data; receiving, at one or more processors, the sequence read data for the plurality of sequence reads; determining, by the one or more processors and based on the sequence read data, an expression level of a first allele of a gene in the sample; determining, by the one or more processors and based on the sequence read data, an expression level of a second allele of the gene in the sample; determining, by the one or more processors, a discrepancy between the expression level of the first allele of the gene and the expression level of the second allele of the gene; predicting that an immunotherapy will be effective to treat a cancer of the subject based at least in part on determining that the discrepancy between the expression level of the first allele of the gene and the expression level of the second allele of the gene is below a threshold; and based on predicting that the immunotherapy will be effective to treat the cancer of the subject, administering the immunotherapy to the subject.
2. The method of clause 1, wherein the sample includes tumor cells and/or circulating tumor cells of the subject.
3. The method of clause 1 or 2, wherein the expression level of the first allele includes a number of the sequence reads having at least one first sequence corresponding to the first allele, and wherein the expression level of the second allele includes a number of the sequence reads having at least one second sequence corresponding to the second allele.
4. The method of any of clauses 1-3, wherein determining, by the one or more processors, the discrepancy between the expression level of the first allele of the gene and the expression level of the second allele of the gene includes: determining that the first allele is a minor allele by determining that the expression level of the first allele in the sample is less than the expression level of the second allele in the sample; and determining a ratio of the expression level of the first allele with respect to: the expression level of the second allele in the sample; or a sum of the expression level of the first allele in the sample and the expression level of the second allele in the sample, and wherein predicting that the immunotherapy will be effective to treat the cancer of the subject includes determining that the ratio is above a ratio threshold.
5. The method of any of clauses 1-4, the gene being a first gene, the method further including: determining, by the one or more processors and based on the sequence read data, at least one expression level of at least one first allele of at least one second gene in the sample; determining, by the one or more processors and based on the sequence read data, at least one expression level of at least one second allele of the at least one second gene in the sample; and determining, by the one or more processors, at least one discrepancy between the at least one expression level of the at least one first allele of the at least one second gene and the expression level of the at least one second allele of the at least one second gene, wherein predicting that the immunotherapy will be effective to treat the cancer of the subject is further based on the at least one discrepancy between the at least one expression level of the at least one first allele of the at least one second gene and the expression level of the at least one second allele of the at least one second gene.
6. The method of clause 5, wherein the first gene and the at least one second gene are selected from: HLA-A, HLA-B, HLA-C, HLA-DP, HLA-DQ, or HLA-DR, and wherein the immunotherapy includes an immune checkpoint inhibitor, a T-cell receptor (TCR) therapy, a TCR-bispecific molecule, or a personalized cancer vaccine.
7. The method of any of clauses 1-6, further including: determining a predicted progression of a tumor of the subject based at least in part on determining that the discrepancy between the expression level of the first allele of the gene and the expression level of the second allele of the gene is below the threshold; and outputting a report indicating the predicted progression of the tumor.
8. A method including: identifying sequence read data representing RNA in a sample obtained from a subject; determining, based on the sequence read data, a discrepancy between expression of a first allele of a gene in the sample and of a second allele of the gene in the sample; and predicting, based at least in part on the discrepancy between the expression of the first allele of the gene in the sample and of the second allele of the gene in the sample, whether a therapy would be effective at treating a cancer of the subject.
9. The method of clause 8, wherein the sequence read data corresponds to a single genomic locus.
10. The method of clause 8 or 9, wherein the sequence read data corresponds to multiple genomic loci.
11. The method of any of clauses 8-10, wherein the sequence read data indicates an RNA transcriptome of the sample.
12. The method of any of clauses 8-11, wherein the sequence read data indicates a whole exome of the sample.
13. The method of any of clauses 8-12, further including: identifying proteomics data of the sample obtained from the subject, wherein the proteomics data indicates a whole proteome and/or a whole peptidome of the sample, wherein determining the discrepancy between the expression of the first allele of the gene in the sample and the second allele of the gene in the sample is further based on the proteomics data.
14. The method of any of clauses 8-13, wherein the gene is associated with chromosome 6.
15. The method of any of clauses 8-14, wherein the sequence read data indicates a predetermined panel of genes of the sample, the predetermined panel including the gene.
16. The method of clause 15, wherein the predetermined panel includes one or more of HLA-A, HLA-B, HLA-C, HLA-D, HLA-E, HLA-F, HLA-G, HLA-DP, HLA-DPA1, HLA-DPA2, HLA-DPB1, HLA-DPB2, HLA-DQ, HLA-DQA1, HLA-DQA2, HLA-DQB1, HLA-DQB2, HLA-DQB3, HLA-DRA, HLA-DRB1, HLA-DRB2, HLA-DRB3, HLA-DRB4, HLA-DRB5, HLA-DM, HLA-DMA, HLA-DMB, HLA-DO, HLA-DOA, or HLA-DOB.
17. The method of any of clauses 8-16, further including: receiving the RNA obtained from the sample; generating a plurality of nucleic acid molecules by reverse transcribing the RNA; ligating one or more adapters onto one or more nucleic acid molecules from the plurality of nucleic acid molecules; amplifying the one or more ligated nucleic acid molecules; capturing all or a subset of the amplified nucleic acid molecules; and sequencing, by a sequencer, the captured nucleic acid molecules to obtain a plurality of sequence reads that represent the captured nucleic acid molecules, thereby generating the sequence read data for a genome of the sample.
18. The method of clause 17, wherein the one or more adapters include amplification primers, flow cell adapter sequences, substrate adapter sequences, or sample index sequences.
19. The method of clause 17 or 18, wherein the captured nucleic acid molecules are captured from the amplified nucleic acid molecules by hybridization to bait molecules.
20. The method of clause 19, wherein the bait molecules include multiple first bait molecules associated with the first allele of the gene, and wherein the bait molecules include multiple second bait molecules associated with the second allele of the gene.
21. The method of clause 19 or 20, wherein the bait molecules include one or more additional nucleic acid molecules, each of the one or more additional nucleic acid molecules including a region that is complementary to a region of a captured nucleic acid molecule.
22. The method of any of clauses 17-21, wherein generating the plurality of nucleic acid molecules by reverse transcribing the RNA includes: synthesizing, by reverse transcriptase, complementary DNA (cDNA) that is complementary to the RNA in the sample, wherein the plurality of nucleic acid molecules includes the cDNA.
23. The method of any of clauses 17-22, wherein amplifying the one or more ligated nucleic acid molecules includes performing a polymerase chain reaction (PCR) amplification technique, a non-PCR amplification technique, an isothermal amplification technique, or a linear antisense RNA (aRNA) amplification technique.
24. The method of any of clauses 17-23, wherein sequencing the captured nucleic acid molecules includes use of whole transcriptome sequencing, a massively parallel sequencing (MPS) technique, whole genome sequencing (WGS), whole exome sequencing, targeted sequencing, direct sequencing, or Sanger sequencing.
25. The method of any of clauses 17-24, wherein sequencing the captured nucleic acid molecules includes next-generation sequencing (NGS).
26. The method of any of clauses 17-25, wherein the sequencer includes a next-generation sequencer.
27. The method of any of clauses 17-26, wherein sequencing the captured nucleic acid molecules includes sequencing-by-synthesis or nanopore sequencing.
28. The method of any of clauses 17-27, wherein the expression of the first allele of the gene includes a number of the sequence reads having at least one first sequence corresponding to the first allele, and wherein the expression of the second allele of the gene includes a number of the sequence reads having at least one second sequence corresponding to the second allele.
29. The method of any of clauses 8-28, further including: generating nucleic acid molecules based on the RNA of the sample; generating ligated molecules by ligating adapters onto the nucleic acid molecules; generating amplified ligated molecules by amplifying the ligated molecules; generating, using the amplified ligated molecules, detection signals; detecting, by at least one sensor, the detection signals; and
generating the sequence read data based on the detection signals.
30. The method of clause 29, wherein the detection signals include electrical signals and/or optical signals.
31. The method of clause 29 or 30, wherein generating, using the amplified ligated molecules, the detection signals includes: synthesizing, by a polymerase using fluorescently tagged nucleotide triphosphates (NTPs), a synthesized nucleic acid molecule that is complementary to one of the amplified ligated molecules, and wherein detecting, by the at least one sensor, the detection signals includes: detecting, by at least one optical sensor, optical signals emitted by the fluorescently tagged NTPs upon binding to the synthesized nucleic acid molecule, the optical signals being indicative of at least one sequence of the nucleic acid molecules of the sample.
32. The method of any of clauses 29-31, wherein generating, using the amplified ligated molecules, the detection signals includes: directing the amplified ligated molecules through a nanopore extending from a first space to a second space through a substrate, and wherein detecting, by the at least one sensor, the detection signals includes: detecting, by sensors disposed in the first space and the second space, an electrical signal over time, the electrical signal being indicative of at least one sequence of the nucleic acid molecules of the sample.
33. The method of any of clauses 8-32, further including: receiving the sample.
34. The method of any of clauses 8-33, wherein the sample includes a tissue biopsy sample, a liquid biopsy sample, or a normal control.
35. The method of any of clauses 8-34, wherein the sample is obtained from a tumor of the subject.
36. The method of any of clauses 8-35, wherein the sample includes tumor cells and/or circulating tumor cells of the subject.
37. The method of any of clauses 8-36, wherein the sample includes non-cancerous tissue of the sample.
38. The method of any of clauses 8-37, wherein the sample is a liquid biopsy sample and includes blood, plasma, cerebrospinal fluid, sputum, stool, urine, lymphatic fluid, or saliva.
39. The method of any of clauses 8-38, wherein the sample is a liquid biopsy sample and includes circulating tumor cells (CTCs).
40. The method of any of clauses 8-39, wherein the sample is a liquid biopsy sample and includes cell-free RNA (cfRNA), circulating tumor RNA (ctRNA), or any combination thereof.
41. The method of any of clauses 8-40, further including extracting the RNA from the sample.
42. The method of clause 41, wherein the RNA includes messenger RNA, microRNA, or non-coding RNA.
43. The method of clause 41 or 42, further including: extracting DNA from the sample.
44. The method of any of clauses 8-43, further including: extracting proteins and/or peptides from the sample.
45. The method of any of clauses 8-44, wherein determining the discrepancy between the expression of the first allele of the gene in the sample and the second allele of the gene in the sample includes: determining a level of RNA expression and/or protein expression of the first allele of the gene in the sample; and determining a level of RNA expression and/or protein expression of the second allele of the gene in the sample.
46. The method of clause 45, wherein determining the level of protein expression of the first allele of the gene includes: extracting proteins corresponding to expression of the first allele from the sample; digesting the proteins in the sample using an enzyme; ionizing the digested proteins by electrospray ionization (ESI), matrix assisted laser desorption ionization (MALDI), atmospheric pressure chemical ionization (APCI), chemical ionization (CI), or electron impact (EI); determining, based on the ionizing the digested proteins, a mass-to-charge ratio of the digested proteins; fragmenting the digested proteins; and analyzing the mass-to-charge ratio of the digested proteins and ions associated with the fragmented proteins.
47. The method of any of clauses 8-46, wherein determining the discrepancy between the expression of the first allele of the gene in the sample and of the second allele of the gene in the sample includes: comparing an expression level of the first allele of the gene to an expression level of the second allele of the gene.
48. The method of any of clauses 8-47, wherein determining the discrepancy between the expression of the first allele of the gene in the sample and of the second allele of the gene in the sample includes: determining that the first allele is a minor allele by determining that the expression of the first allele in the sample is less than the expression of the second allele in the sample; and determining a ratio of the expression of the first allele with respect to: the expression of the second allele in the sample; or a sum of the expression of the first allele in the sample and the expression of the second allele in the sample.
49. The method of clause 48, wherein predicting whether the therapy would be effective at treating the cancer of the subject includes: predicting that the therapy will be effective to treat the cancer of the subject based at least in part on determining that the ratio is above a threshold.
50. The method of clause 48 or 49, wherein predicting whether the therapy would be effective at treating the cancer of the subject includes: predicting that the therapy will be ineffective to treat the cancer of the subject based at least in part on determining that the ratio is below a threshold.
51. The method of any of clauses 48-50, wherein the ratio is in a range of about 0.01 to about 0.5.
52. The method of any of clauses 48-51, wherein the ratio is in a range of about 0.2 to 0.4.
53. The method of any of clauses 8-52, further including: determining homozygous expression of the gene by determining that the discrepancy between the expression of the first allele of the gene and of the second allele of the gene is above a threshold.
54. The method of clause 53, further including: determining, based on the sequence read data, homozygous DNA associated with the gene.
55. The method of clause 53 or 54, further including: determining, based on the sequence read data, heterozygous DNA associated with the gene.
56. The method of any of clauses 8-55, further including: determining heterozygous expression of the gene by determining that the discrepancy between the expression of the first allele of the gene and the expression of the second allele of the gene is below a threshold.
57. The method of any of clauses 8-56, wherein the gene includes an HLA class I gene, an HLA class II gene, HLA-A, HLA-B, HLA-C, HLA-D, HLA-E, HLA-F, HLA-G, HLA-DP, HLA-DPA1, HLA-DPA2, HLA-DPB1, HLA-DPB2, HLA-DQ, HLA-DQA1, HLA-DQA2, HLA-DQB1, HLA-DQB2, HLA-DQB3, HLA-DRA, HLA-DRB1, HLA-DRB2, HLA-DRB3, HLA-DRB4, HLA-DRB5, HLA-DM, HLA-DMA, HLA-DMB, HLA-DO, HLA-DOA, or HLA-DOB.
58. The method of any of clauses 8-57, further including: determining, based on the discrepancy between the expression of the first allele of the gene in the sample and of the second allele of the gene in the sample, input features; and determining, using a model and based on the input features, an expression condition indicative of whether the therapy would effectively treat the cancer of the subject.
59. The method of clause 58, wherein the input features are determined based at least in part on pre-classified data, the pre-classified data being generated by: identifying training sequence read data associated with samples corresponding to a plurality of individuals omitting the subject; and generating the pre-classified data by labeling the training sequence read data with labels indicative of expression conditions of the plurality of individuals.
60. The method of clause 59, further including: training a machine learning (ML) model to identify attributes, indicated by the training sequence read data, that are predictive of the expression conditions of the plurality of individuals, wherein the input features are instances of the attributes identified via the training of the ML model.
61. The method of clause 59 or 60, wherein the pre-classified data is based on a sample obtained from an individual that has the expression condition.
62. The method of any of clauses 59-61, wherein the pre-classified data is based on a sample obtained from an individual that lacks the expression condition.
63. The method of any of clauses 58-62, wherein determining the input features includes: identifying RNA sequences and/or DNA sequences associated with the first allele of the gene and with the second allele of the gene in the sample.
64. The method of any of clauses 58-63, wherein determining, using a model and based on the input features, the expression condition indicative of whether the therapy would effectively treat the cancer of the subject includes: generating a classification of the expression condition by inputting, into a classifier, the input features.
65. The method of clause 64, wherein the classifier includes an ML-based classifier.
66. The method of clause 65, further including: training the ML-based classifier using training data, the training data including population features of population samples obtained from a population omitting the subject.
67. The method of clause 66, wherein training the ML-based classifier using the training data includes performing supervised learning on the ML-based classifier.
68. The method of clause 66 or 67, wherein training the ML-based classifier using the training data includes performing unsupervised learning on the ML-based classifier.
69. The method of any of clauses 66-68, wherein training the ML-based classifier using the training data includes optimizing parameters of the ML-based classifier using the training data.
70. The method of any of clauses 65-69, wherein the ML-based classifier includes at least one of a: an artificial neural network (ANN); a logistic regression model; a random forest model; a decision tree; a k-nearest neighbor (KNN) model; a support vector machine (SVM); or a naïve Bayes classifier.
71. The method of any of clauses 8-70, further including: determining endpoint counts of DNA fragments and/or RNA fragments indicated by the sequence read data, wherein predicting whether the therapy would be effective at treating the cancer of the subject is based at least in part on the endpoint counts of the DNA fragments and/or the RNA fragments indicated by the sequence read data.
72. The method of clause 71, wherein the endpoint counts include left endpoint counts and/or right endpoint counts.
73. The method of any of clauses 8-72, wherein the gene is a first gene and the discrepancy is a first discrepancy, the method further including: determining, based on the sequence read data, at least one expression of at least one first allele of at least one second gene in the sample; determining, based on the sequence read data, at least one expression of at least one second allele of the at least one second gene in the sample; and determining at least one second discrepancy between the at least one expression of the at least one first allele of the at least one second gene and the expression of the at least one second allele of the at least one second gene.
74. The method of clause 73, further including: determining that the first discrepancy is greater than a first threshold; determining that the at least one second discrepancy is lower than a second threshold; and determining, based on the first discrepancy and the at least one second discrepancy, that the therapy would be effective in treating the cancer of the subject.
75. The method of clause 73 or 74, further including: determining that the first discrepancy is lower than a first threshold; determining that the at least one second discrepancy is greater than a second threshold; and determining, based on the first discrepancy and the at least one second discrepancy, that the therapy would be ineffective at treating the cancer of the subject.
76. The method of any of clauses 73-75, further including: determining that the first discrepancy is lower than a first threshold; determining that the at least one second discrepancy is lower than a second threshold; and determining, based on the first discrepancy and the at least one second discrepancy, that the therapy would be effective at treating the cancer of the subject.
77. The method of any of clauses 73-76, further including: determining that the first discrepancy is greater than a first threshold; determining that the at least one second discrepancy is greater than a second threshold; and determining, based on the first discrepancy and the at least one second discrepancy, that the therapy would be ineffective at treating the cancer of the subject.
78. The method of any of clauses 73-77, wherein the first gene and the at least one second gene are selected from HLA-A, HLA-B, HLA-C, HLA-D, HLA-E, HLA-F, HLA-G, HLA-DP, HLA-DPA1, HLA-DPA2, HLA-DPB1, HLA-DPB2, HLA-DQ, HLA-DQA1, HLA-DQA2, HLA-DQB1, HLA-DQB2, HLA-DQB3, HLA-DRA, HLA-DRB1, HLA-DRB2, HLA-DRB3, HLA-DRB4, HLA-DRB5, HLA-DM, HLA-DMA, HLA-DMB, HLA-DO, HLA-DOA, or HLA-DOB.
79. The method of any of clauses 8-78, wherein the cancer is bladder cancer, breast cancer, colorectal cancer, a melanoma, non-small cell lung cancer (NSCLC), pancreatic cancer, or prostate cancer.
80. The method of any of clauses 8-79, wherein the cancer is a B cell cancer (multiple myeloma), a melanoma, breast cancer, lung cancer, NSCLC, small cell lung cancer (SCLC), bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms'tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
81. The method of any of clauses 8-80, further including: determining, based at least in part on the discrepancy between the expression of the first allele of the gene in the sample and the second allele of the gene in the sample, at least one of: a loss of heterozygosity associated with the gene; a heterozygosity associated with the gene; a loss of function associated with the gene; a metric associated with loss of heterozygosity of the gene; or a metric associated with loss of function of the gene.
82. The method of any of clauses 8-81, further including determining, based at least in part on the discrepancy between the expression of the first allele of the gene in the sample and the second allele of the gene in the sample, at least one of: a cancer type of the subject; a cancer subtype of the subject; a metastasis profile of the subject; a predicted survivability of the subject; a predicted symptom of the subject; a predicted effective therapy to treat the cancer of the subject; a predicted resistance of the subject to a treatment of the cancer; a general health of the subject; a genomic age of the subject; a predicted stage of the cancer of the subject; a predicted grade of the cancer of the subject; or a predicted Eastern Cooperative Oncology Group (ECOG) performance status of the subject.
83. The method of any of clauses 8-82, further including: determining, based at least in part on the discrepancy between the expression of the first allele of the gene in the sample and the second allele of the gene in the sample, a health metric and/or a disease metric of the subject.
84. The method of any of clauses 8-83, further including: generating a genomic profile of the subject.
85. The method of clause 84, wherein the genomic profile includes results from at least one of: a whole transcriptome sequencing test; a targeted RNA sequencing test; a long-read RNA sequencing test; a comprehensive genomic profiling test; a whole genome sequencing (WGS) test; a whole exome sequencing (WES) test; a gene expression profiling test; a cancer hotspot panel test; a DNA methylation test; a DNA fragmentation test; or an RNA fragmentation test.
86. The method of clause 85, further including: determining, based on the DNA methylation test, a DNA methylation profile associated with the gene, wherein determining whether the therapy would effectively treat the cancer of the subject is based at least in part on the DNA methylation profile.
87. The method of any of clauses 84-86, wherein the genomic profile of the subject includes: results from a nucleic acid sequencing-based test.
88. The method of any of clauses 84-87, further including: determining, based on the expression of the first allele of the gene in the sample and of the second allele of the gene in the sample and/or the genomic profile, a type and/or a subtype of the cancer of the subject.
89. The method of any of clauses 84-88, further including: determining, based on the expression of the first allele of the gene in the sample and of the second allele of the gene in the sample and/or the genomic profile, a genomic subtype and/or a phenotypic subtype of the cancer of the subject.
90. The method of any of clauses 84-89, further including: determining, based on the expression of the first allele of the gene in the sample and of the second allele of the gene in the sample and/or the genomic profile, that the therapy would not effectively treat the cancer of the subject; and selecting, based on the expression of the first allele of the gene in the sample and of the second allele of the gene in the sample and/or the genomic profile, a second therapy predicted to effectively treat the cancer of the subject.
91. The method of any of clauses 84-90, further including: determining, based on the expression of the first allele of the gene in the sample and of the second allele of the gene in the sample and/or the genomic profile, that the therapy would effectively treat the cancer of the subject.
92. The method of clause 91, further including: administering, based on determining whether the therapy would effectively treat the cancer of the subject, the therapy to the subject.
93. The method of any of clauses 8-92, wherein the therapy includes at least one of chemotherapy, radiation therapy, immunotherapy, a targeted therapy, or surgery.
94. The method of clause 93, wherein the immunotherapy includes an immune checkpoint inhibitor, a T-cell receptor (TCR) therapy, a TCR-bispecific molecule, or a personalized cancer vaccine.
95. The method of any of clauses 8-94, further including: generating a report indicating whether the therapy would effectively treat the cancer of the subject and/or the expression of the first allele of the gene in the sample and or the second allele of the gene in the sample; and outputting the report.
96. The method of clause 95, wherein outputting the report includes: transmitting data indicating the report to an external device.
97. The method of clause 96, wherein the external device is associated with the subject and/or a healthcare provider.
98. The method of clause 96 or 97, wherein the data is transmitted over one or more communication networks.
99. The method of any of clauses 96-98, wherein the data is transmitted over a peer-to-peer connection.
100. The method of any of clauses 95-99, wherein outputting the report includes: visually presenting, by a display, the report.
101. The method of any of clauses 8-100, wherein the therapy includes a dosage of one or more therapeutic agents predicted to treat the cancer of the subject.
102. The method of any of clauses 8-101, further including: determining, based on the expression of the first allele of the gene in the sample and or the second allele of the gene in the sample, whether the subject is eligible for a clinical trial.
103. A system, including: at least one processor; and memory storing instructions that, when executed by the at least one processor, cause the at least one processor to perform operations including: identifying sequence read data representing RNA in a sample obtained from a subject; determining, based on the sequence read data, a discrepancy between expression of a first allele of a gene in the sample and of a second allele of the gene in the sample; and predicting, based at least in part on the discrepancy between the expression of the first allele of the gene in the sample and the second allele of the gene in the sample, whether a therapy would be effective at treating a cancer of the subject.
104. The system of clause 103, further including: a sequencer configured to generate the sequence read data by sequencing a plurality of nucleic acid molecules in the sample.
105. The system of clause 103 or 104, further including: a transceiver configured to transmit data indicating whether the therapy would effectively treat the cancer of the subject and/or the expression of the first allele of the gene in the sample and of the second allele of the gene in the sample.
106. The system of any of clauses 103-105, further including: an output device configured to output an indication of whether the therapy would effectively treat the cancer of the subject and/or the expression of the first allele of the gene in the sample and of the second allele of the gene in the sample.
107. A non-transitory computer readable medium storing instructions for performing operations including: identifying sequence read data representing RNA in a sample obtained from a subject; determining, based on the sequence read data, a discrepancy between expression of a first allele of a gene in the sample and of a second allele of the gene in the sample; and predicting, based at least in part on the discrepancy between the expression of the first allele of the gene in the sample and the second allele of the gene in the sample, whether a therapy would be effective at treating a cancer of the subject.
108. A method of identifying an individual having a cancer and predicted to respond to a therapy to treat the cancer, the method including detecting in a sample from the individual: a predetermined pattern of expression of a first allele of a gene and of a second allele of the gene in a sample of the individual, wherein detection of predetermined pattern of expression of the first allele of the gene and of the second allele of the gene identifies the individual as one who may respond to the therapy.
109. A method of treating or delaying progression of a cancer in an individual in need thereof, including: acquiring knowledge of: expression of a first allele of a gene and of a second allele of the gene in a sample of the individual; selecting a treatment based on the expression of the first allele of the gene and of the second allele of the gene; and administering to the individual an effective amount of the treatment.

### Conclusion

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, the practice of the present disclosure can employ conventional techniques of immunology, molecular biology, microbiology, cell biology and recombinant DNA. These methods are described in the following publications. See, e.g., Green and Sambrook, Molecular Cloning: A Laboratory Manual, 4nd Edition (2012); F. M. Ausubel, et al. eds., Current Protocols in Molecular Biology, (2003); the series Methods In Enzymology (Academic Press, Inc.); Behlke, et al., Polymerase Chain Reaction: Theory and Technology (2019); Greenfield, ed. Antibodies, A Laboratory Manual, Second Edition (2014); and Capes-Davis and R. I. Freshney, eds. Freshney's Culture of Animal Cells 8th Edition (2021).

Tumor mutational burden (TMB) is a measure of the number of mutations carried by tumor cells. By comparing DNA sequences from a patient's healthy tissues and tumor cells, the number of acquired somatic mutations present in tumors, but not in normal tissues, may be determined. In some instances, driver mutations may be excluded from a TMB calculation.

In certain examples, “tumor mutational burden” or “TMB” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In various cases, driver mutations are excluded from a TMB calculation.

Microsatellites are highly polymorphic DNA-repeat regions. In certain examples, “microsatellite” refers to a repetitive nucleic acid having repeat units of less than about 10 base pairs or nucleotides in length. In certain examples, a microsatellite refers to a tract of tandemly repeated (i.e. adjacent) DNA motifs ranging from one to six or up to ten nucleotides, with each motif repeated 5 to 50 repeated times. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy,” Appl Immunohistochem Mol Morphol, 26 (2): e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma.

A viral status test refers to a test that identifies the presence of viral RNA or DNA in a subject. The test can identify viral load and/or viral identity. For example, the viral status test can identify the presence of viral RNA or DNA associated with the occurrence of certain cancers. Examples of such viruses include Hepatitis B Virus (HBV) and Hepatitis C Virus (HCV), Kaposi Sarcoma-Associated Herpesvirus (KSHV), Merkel Cell Polyomavirus (MCV), Human Papillomavirus (HPV), Human Immunodeficiency Virus Type 1 (HIV-1, or HIV), Human T-Cell Lymphotropic Virus Type 1 (HTLV-1), and Epstein-Barr Virus (EBV).

Cancer “hotspot” mutations give rise to oncological outcomes. PhyloP, SIFT, Grantham, COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation, EGFR exon 20 activation mutation, EGFR exon 20 insertion, EGFR G719 mutation, EGFR L858R mutation, EGFR L861 mutation, EGFR S768 mutation, EGFR T790M mutation, C797 mutation, KIT activating mutation, KRAS activating mutation, MET activating mutation, NRAS activating mutation, PMS2 promoter mutations, among many others. Hotspot mutations also occur in the following genes: AKT2, BRCA1, BRCA2, ERC1, NSD1, POLH, PPM1G, PTEN, RAD18, RAD51, RAD51B, RB1, TERT, TP53, TP53Bp1, ALK, ARMT1, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

A “DNA methylation test” refers to an assay, which can be commercially available, for distinguishing methylated versus unmethylated cytosine loci in DNA. Techniques for measuring cytosine methylation include bisulfite-based methylation assays. The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, CA) a quantitative methylation detection assay that uses fluorescence-based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify the target sequence of interest. The amplified PCR product is isolated and used to quantitate the methylation status of the CpG site of interest. (Gonzalgo and Jones Nuclei Acids Res1997; 25:252-31).

In particular embodiments, pyrosequencing can be used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343:249-264. Pyrosequencing is especially useful for detecting methylation in the CpG sites within genes.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Read depth refers to the number of times that a specific genomic site is sequenced during a sequencing run.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A method, comprising:
providing a plurality of RNA molecules obtained from a sample from a subject;
generating a plurality of nucleic acid molecules by reverse transcribing the RNA molecules;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, all or a subset of the captured amplified nucleic acid molecules to obtain a plurality of sequence reads that represent the sequenced amplified nucleic acid molecules thereby generating sequence read data;
receiving, at one or more processors, the sequence read data for the plurality of sequence reads;
determining, by the one or more processors and based on the sequence read data, an expression level of a first allele of a gene in the sample;
determining, by the one or more processors and based on the sequence read data, an expression level of a second allele of the gene in the sample;
determining, by the one or more processors, a discrepancy between the expression level of the first allele of the gene and the expression level of the second allele of the gene;
predicting that an immunotherapy will be effective to treat a cancer of the subject based at least in part on determining that the discrepancy between the expression level of the first allele of the gene and the expression level of the second allele of the gene is below a threshold; and
based on predicting that the immunotherapy will be effective to treat the cancer of the subject, administering the immunotherapy to the subject.

2. The method of claim 1, wherein the sample comprises tumor cells and/or circulating tumor cells of the subject.

3. The method of claim 1, wherein the expression level of the first allele comprises a number of the sequence reads having at least one first sequence corresponding to the first allele, and
wherein the expression level of the second allele comprises a number of the sequence reads having at least one second sequence corresponding to the second allele.

4. The method of claim 1, wherein determining, by the one or more processors, the discrepancy between the expression level of the first allele of the gene and the expression level of the second allele of the gene comprises:
determining that the first allele is a minor allele by determining that the expression level of the first allele in the sample is less than the expression level of the second allele in the sample; and
determining a ratio of the expression level of the first allele with respect to:
the expression level of the second allele in the sample; or
a sum of the expression level of the first allele in the sample and the expression level of the second allele in the sample, and
wherein predicting that the immunotherapy will be effective to treat the cancer of the subject comprises determining that the ratio is above a ratio threshold.

5. The method of claim 1, the gene being a first gene, the method further comprising:
determining, by the one or more processors and based on the sequence read data, at least one expression level of at least one first allele of at least one second gene in the sample;
determining, by the one or more processors and based on the sequence read data, at least one expression level of at least one second allele of the at least one second gene in the sample; and
determining, by the one or more processors, at least one discrepancy between the at least one expression level of the at least one first allele of the at least one second gene and the expression level of the at least one second allele of the at least one second gene,
wherein predicting that the immunotherapy will be effective to treat the cancer of the subject is further based on the at least one discrepancy between the at least one expression level of the at least one first allele of the at least one second gene and the expression level of the at least one second allele of the at least one second gene.

6. The method of claim 5, wherein the first gene and the at least one second gene are selected from: HLA-A, HLA-B, HLA-C, HLA-DP, HLA-DQ, or HLA-DR, and
wherein the immunotherapy comprises an immune checkpoint inhibitor, a T-cell receptor (TCR) therapy, a TCR-bispecific molecule, or a personalized cancer vaccine.

7. The method of claim 1, further comprising:
determining a predicted progression of a tumor of the subject based at least in part on determining that the discrepancy between the expression level of the first allele of the gene and the expression level of the second allele of the gene is below the threshold; and
outputting a report indicating the predicted progression of the tumor.

8. A method comprising:
identifying sequence read data representing RNA in a sample obtained from a subject;
determining, based on the sequence read data, a discrepancy between expression of a first allele of a gene in the sample and of a second allele of the gene in the sample; and
predicting, based at least in part on the discrepancy between the expression of the first allele of the gene in the sample and of the second allele of the gene in the sample, whether a therapy would be effective at treating a cancer of the subject.

9. The method of claim 8, wherein determining the discrepancy between the expression of the first allele of the gene in the sample and the second allele of the gene in the sample comprises:
determining a level of RNA expression and/or protein expression of the first allele of the gene in the sample; and
determining a level of RNA expression and/or protein expression of the second allele of the gene in the sample.

10. The method of claim 8, wherein determining the discrepancy between the expression of the first allele of the gene in the sample and of the second allele of the gene in the sample comprises:
determining that the first allele is a minor allele by determining that the expression of the first allele in the sample is less than the expression of the second allele in the sample;
determining a ratio of the expression of the first allele with respect to:
the expression of the second allele in the sample; or
a sum of the expression of the first allele in the sample and the expression of the second allele in the sample; and
predicting whether the therapy will be effective to treat the cancer of the subject based at least in part on comparing the ratio to a threshold.

11. The method of claim 10, wherein the ratio is in a range of about 0.01 to about 0.5.

12. The method of claim 8, further comprising:
determining homozygous expression or heterozygous expression of the gene by determining whether the discrepancy between the expression of the first allele of the gene and of the second allele of the gene is above a threshold.

13. The method of claim 8, wherein the gene comprises an HLA class I gene, an HLA class II gene, HLA-A, HLA-B, HLA-C, HLA-D, HLA-E, HLA-F, HLA-G, HLA-DP, HLA-DPA1, HLA-DPA2, HLA-DPB1, HLA-DPB2, HLA-DQ, HLA-DQA1, HLA-DQA2, HLA-DQB1, HLA-DQB2, HLA-DQB3, HLA-DRA, HLA-DRB1, HLA-DRB2, HLA-DRB3, HLA-DRB4, HLA-DRB5, HLA-DM, HLA-DMA, HLA-DMB, HLA-DO, HLA-DOA, or HLA-DOB.

14. The method of claim 8, further comprising:
determining endpoint counts of DNA fragments and/or RNA fragments indicated by the sequence read data,
wherein predicting whether the therapy would be effective at treating the cancer of the subject is based at least in part on the endpoint counts of the DNA fragments and/or the RNA fragments indicated by the sequence read data.

15. The method of claim 8, wherein the gene is a first gene and the discrepancy is a first discrepancy, the method further comprising:
determining, based on the sequence read data, at least one expression of at least one first allele of at least one second gene in the sample;
determining, based on the sequence read data, at least one expression of at least one second allele of the at least one second gene in the sample;
determining at least one second discrepancy between the at least one expression of the at least one first allele of the at least one second gene and the expression of the at least one second allele of the at least one second gene;
determining whether the first discrepancy is greater than a first threshold;
determining whether the at least one second discrepancy is lower than a second threshold; and
determining, based on whether the first discrepancy is greater than the first threshold and whether the at least one second discrepancy is greater than the second threshold, whether the therapy would be effective in treating the cancer of the subject.

16. The method of claim 8, wherein the cancer is a B cell cancer (multiple myeloma), a melanoma, breast cancer, lung cancer, NSCLC, small cell lung cancer (SCLC), bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms'tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

17. The method of claim 8, further comprising: determining, based at least in part on the discrepancy between the expression of the first allele of the gene in the sample and the second allele of the gene in the sample, at least one of:
a loss of heterozygosity associated with the gene;
a heterozygosity associated with the gene;
a loss of function associated with the gene;
a metric associated with loss of heterozygosity of the gene; or
a metric associated with loss of function of the gene.

18. The method of claim 8, further comprising determining, based at least in part on the discrepancy between the expression of the first allele of the gene in the sample and the second allele of the gene in the sample, at least one of:
a cancer type of the subject;
a cancer subtype of the subject;
a metastasis profile of the subject;
a predicted survivability of the subject;
a predicted symptom of the subject;
a predicted effective therapy to treat the cancer of the subject;
a predicted resistance of the subject to a treatment of the cancer;
a general health of the subject;
a genomic age of the subject;
a predicted stage of the cancer of the subject;
a predicted grade of the cancer of the subject; or
a predicted Eastern Cooperative Oncology Group (ECOG) performance status of the subject.

19. The method of claim 8, wherein the therapy comprises at least one of chemotherapy, radiation therapy, immunotherapy, a targeted therapy, or surgery, and
wherein the immunotherapy comprises an immune checkpoint inhibitor, a T-cell receptor (TCR) therapy, a TCR-bispecific molecule, or a personalized cancer vaccine.

20. A method of treating or delaying progression of a cancer in an individual in need thereof, comprising:
acquiring knowledge of:
expression of a first allele of a gene and of a second allele of the gene in a sample of the individual;
selecting a treatment based on the expression of the first allele of the gene and of the second allele of the gene; and
administering to the individual an effective amount of the treatment.
